(19) United States
(12) Patent Application Publication
(10) Pub. No.: US 2025/0144411 A1
(43) Pub. Date: May 8, 2025

(54) STIMULATOR DEVICE FOR PROVIDING A STIMULATION SIGNAL, A SYSTEM FOR PROVIDING TEMPORAL INTERFERENCE STIMULATION, AND A METHOD FOR MONITORING A STIMULATION SIGNAL

(71) Applicant: Stichting IMEC Nederland, Eindhoven (NL)

(72) Inventors: Haoming XIN, Eindhoven (NL); Roland VAN WEGBERG, Oss (NL); Meiyi ZHOU, Eindhoven (NL); Mark FICHMAN, Eindhoven (NL)

(21) Appl. No.: 18/931,153

(22) Filed: Oct. 30, 2024

(30) Foreign Application Priority Data
Nov. 3, 2023 (EP) 23207588.7

(51) Int. Cl. A61N 1/32; A61N 1/36

(52) U.S. Cl. CPC A61N 1/323; A61N 1/36153; A61N 1/36157

(57) ABSTRACT

A stimulator device for providing a stimulation signal comprises: a stimulation signal generating unit configured to generate a stimulation current signal and provide the stimulation current signal to an output of the stimulation signal generating unit, wherein the stimulation current signal comprises a sinusoidal-like waveform; and an impedance monitoring unit connected to the output of the stimulation signal generating unit, wherein the impedance monitoring unit is configured to, while the stimulation current signal is provided on the output of the stimulation signal generating unit, determine an extreme voltage at an extreme point of a voltage signal at the output and a phase delay between the voltage signal and the stimulation current signal for determining information of an impedance relating to an interface between an electrode, connected to the output, and tissue.

### CROSS-REFERENCE TO RELATED APPLICATIONS

The present application claims the benefit of and priority to EP patent application Ser. No. 23/207,588.7, filed Nov. 3, 2023, the entire contents of which is incorporated herein by reference.

### TECHNICAL FIELD

The present description relates to a stimulator device and, in particular, a stimulator device which may be part of a system for providing temporal interference stimulation of a brain and/or a nerve. The present description also relates to a method for monitoring a stimulation signal.

### BACKGROUND

Electrical stimulation is widely used in clinical practice. Stimulation may typically be delivered using biphasic charge balanced pulses.

When an accurate spatial selectivity of stimulation is desired, interferential stimulation based on two or more stimulation signals may be used. Interference of the two or more stimulation signals may thus generate an interferential stimulation signal within tissue, which allows selectively stimulating a location within the tissue. The stimulation signals for forming the interferential stimulation may be continuously provided for a long period of time.

An interface between an electrode and tissue, for providing a stimulation signal into the tissue, may change over time. This may affect the electrical stimulation and/or relate to safety issues for providing the electrical stimulation.

Thus, there is a need to acquire information of the electrode-tissue interface and, in particular, to acquire information during output of a stimulation signal since the stimulation signal may be continuously provided for a long period of time.

### SUMMARY

An objective of the present description is to enable acquiring of information of an electrode-tissue interface used for providing a stimulation signal into tissue. A particular objective of the present description is to enable acquiring of the information during output of the stimulation signal.

Another objective of the present description is to enable an improved control of interferential stimulation. A particular objective of the present description is to facilitate blocking propagation of neural signals using interferential stimulation.

These and other objectives are at least partly met by the invention as defined in the independent claims. Preferred embodiments are set out in the dependent claims.

According to a first aspect, there is provided a stimulator device for providing a stimulation signal, said stimulator device comprising: a stimulation signal generating unit configured to generate a stimulation current signal and provide the stimulation current signal to an output of the stimulation signal generating unit, wherein the stimulation current signal comprises a sinusoidal-like waveform; and an impedance monitoring unit connected to the output of the stimulation signal generating unit, wherein the impedance monitoring unit is configured to, while the stimulation current signal is provided on the output of the stimulation signal generating unit, determine an extreme voltage at an extreme point of a voltage signal at the output and a phase delay between the voltage signal and the stimulation current signal for determining information of an impedance relating to an interface between an electrode, connected to the output, and tissue.

Thanks to the stimulator device of the first aspect, impedance of the electrode-tissue interface can be determined. The information of the impedance is determined based on the impedance monitoring unit being connected to the output of the stimulation signal generating unit. Thus, there is no need for additional electrodes for acquiring signals that are to be used for determining the impedance. Rather, the impedance may be directly determined at the output of the stimulation signal generating unit.

In addition, the stimulation signal generating unit is configured to generate a current signal, whereas the impedance monitoring unit is configured to determine a voltage. Thus, the impedance monitoring unit may determine the voltage while the stimulation current signal is provided on the output of the stimulation generating unit. In particular, the stimulator device is configured to utilize the stimulation current signal for determining information of impedance, such that a dedicated signal for determining impedance need not be used but rather the stimulation current signal used for providing a stimulation may also be used for determining information of impedance. Thus, information of impedance may be determined in an energy efficient manner.

The extreme voltage at the extreme point of the voltage signal and the phase delay between the voltage signal and the stimulation current signal may be used for determining a complex impedance (including resistive and capacitive values) of the interface between the electrode and tissue. The determining of the complex impedance may further use known information of characteristics of the stimulation current signal, such as using information of amplitude and carrier frequency of the stimulation current signal.

The information of the impedance of the interface between the electrode and tissue may be used in numerous ways. The impedance may provide clinical information to a doctor. For instance, for an implanted device, tissue may grow around an electrode that is implanted in a body. The impedance of the interface between the electrode and tissue may thus provide a measure relating to tissue growth around the electrode.

The impedance between the electrode and tissue will affect a supply voltage needed by the stimulation signal generating unit for providing a desired amplitude of the stimulation current signal. The information of the impedance may thus be used for controlling a supply voltage so as to allow saving power in generating of the stimulation current signal.

Thus, thanks to the determined impedance information, a closed feedback loop may be provided during stimulation for controlling the supply voltage. The supply voltage may be set to a needed level with some minor margin to ensure that the desired amplitude of the stimulation current signal is achieved, while not using an unnecessarily high level of the supply voltage. This implies that the stimulation current signal may be generated in a power efficient manner.

The information of the impedance of the interface between the electrode and tissue may be used for determining whether the electrode is properly arranged in contact with tissue. Thus, information of impedance may be used for monitoring that the electrode is properly arranged and may be used for identifying if the electrode is affected so as to lose proper contact with tissue.

It should further be realized that even though information of impedance may be acquired during stimulation, impedance information may also be acquired in a separate measurement. The impedance information may be acquired using a low excitation current signal being output to electrodes for determining the impedance. This may be used for monitoring electrode-tissue interface to ensure that the electrode maintains proper contact with tissue. The use of a low excitation current allows the measurement to be performed without or with minimal discomfort to a patient.

In addition, impedance between an electrode and a nerve may be slightly affected when an action potential is triggered inside the nerve. Thus, by monitoring information of the impedance of the interface between the electrode and tissue, the stimulator device may be able to determine if the stimulation current signal is successful in triggering an action potential in the nerve. This may further be used for adjusting control of a stimulation current signal to be output by the stimulation signal generating unit.

The stimulator device may be particularly useful for generating stimulation signals that are to be used for temporal interference stimulation, wherein multiple stimulation signals interfere for causing an interferential signal within a brain and/or a nerve to be stimulated, such that the interferential signal may provide a desired stimulation. Thus, a plurality of stimulator devices may be used, each dedicated to generating one stimulation signal that is to be used for forming the temporal interference stimulation. The temporal interference stimulation allows spatially selecting a location to be stimulated, such as a location in a brain and/or a nerve of a living being, such as a human being or an animal.

The stimulation current signal having a sinusoidal-like waveform implies that the stimulation current signal may be sinusoidal but does not necessarily need to be a pure sinusoidal waveform. For instance, the stimulation current signal may be a pseudo-sinusoidal waveform, a modified sinusoidal waveform, or a multi-level waveform approximating a sinusoidal waveform. The stimulation current signal may further be an alternating current (AC) signal.

The stimulation current signal may be a continuous signal, which is provided over a relatively long period of time. However, the stimulation current signal may be a sequence of temporally separated bursts, wherein each burst comprises the sinusoidal-like waveform. Using bursts, power consumption may be limited since the stimulation current signal need not be constantly output, while a corresponding effect on tissue, such as a brain and/or a nerve, of the stimulation signal compared to a continuous stimulation signal may still be achieved.

The stimulation current signal may be used for stimulating a brain and/or a nerve of a living being. For instance, the stimulation current signal may be used for triggering a neural signal to be generated in the brain and/or the nerve or for blocking a neural signal that is propagating in the brain and/or the nerve. Temporal interferential stimulation may be used for accurately controlling a spatial location in the brain and/or the nerve in which the stimulation is provided.

The stimulation signal generating unit may output a current signal. The current signal may be converted to a voltage signal by being output to an electrode connected to tissue. Thus, the impedance monitoring unit may be configured to monitor a voltage signal based on a stimulation signal which is a current signal.

The voltage signal varies with time and comprises extreme points in a time-dependent signal. An extreme point is a point of the voltage signal at a particular time instance when the voltage signal has a local maximum value or local minimum value, and a time derivative of the voltage signal is zero. The extreme voltage is thus a voltage value at the extreme point.

The extreme voltage may be referred to herein as a peak voltage, which corresponds to a local maximum value of the voltage signal. However, it should be realized that, when discussion of peak voltage is made herein, a local minimum value of the voltage signal may alternatively be used.

According to an embodiment, the stimulator device further comprises an electrode connected to the output of the signal generating unit for receiving the stimulation current signal, said electrode being configured for connection to tissue for providing the stimulation current signal into a body part.

The electrode may be configured to be implanted, such that the electrode may be connected to tissue within a living being, such as connected to a wall of a nerve within the living being. However, the electrode may alternatively be configured to be arranged in contact with the living being, externally to the living being, such as arranged in contact with skin. It should be further realized that the electrode may not necessarily be in direct contact with the tissue. For instance, a gel may be provided between the electrode and the tissue in order to improve electrical contact between the electrode and the tissue.

The body part may for instance be a brain and/or a nerve of a living being, such that the stimulator device is configured to provide the stimulation signal for stimulating the brain and/or the nerve. It should be realized that even though reference is made herein to the brain and/or the nerve being stimulated, the stimulator device may also or alternatively be configured for stimulating a spinal cord, or a dorsal route ganglion. It should be realized that the stimulation signal generating unit and the impedance monitoring unit may be packaged together for providing generation of a stimulation current signal and impedance monitoring within a compact device. The stimulator device may further comprise the electrode, such that the stimulation signal generating unit and the impedance monitoring unit may be delivered together with the electrode. However, it should be realized that the electrode may alternatively be separately delivered and that the stimulation signal generating unit and the impedance monitoring unit may be connected to the electrode when the stimulator device is to be used.

According to an embodiment, the stimulator device further comprises a processor configured to receive the extreme voltage and the phase delay and configured to determine the impedance based on the received extreme voltage and phase delay and knowledge of an amplitude and frequency of the stimulation current signal.

This implies that the stimulator device is configured to determine the impedance within a processor that is part of the stimulator device. For instance, the processor may be arranged in a common housing with the stimulation signal generating unit and the impedance monitoring unit. This may ensure that the impedance may be determined within a compact device and that impedance may be determined in a processor that is physically close to the stimulation signal generating unit. Hence, use of the determined impedance for real-time control of the stimulator device is facilitated.

The processor may have knowledge of the amplitude and frequency of the stimulation signal. Information of the amplitude and frequency of the stimulation signal may be stored such that the processor has access to the information.

The processor may be configured to provide a control signal to the stimulation signal generating unit. This implies that the processor has knowledge of the amplitude and frequency of the stimulation signal based on the processor being configured to control these characteristics of the stimulation signal being generated by the stimulation signal generating unit.

The processor and the stimulation signal generating unit may be configured to communicate with each other. Thus, the stimulation signal generating unit may communicate information of the amplitude and frequency of the stimulation signal to the processor.

The stimulation signal generating unit, the impedance monitoring unit, the electrode, and the processor may be arranged in a compact device. The compact device may thus be able to generate the stimulation signal, provide an interface for output of the stimulation signal to tissue, and determine the impedance of the interface. However, according to an alternative, the stimulation signal generating unit and the impedance monitoring unit, the electrode, and the processor may form parts of a stimulator system, wherein components are arranged in separate physical units.

According to an embodiment, the stimulator device further comprises a compensation unit configured to generate a compensation current signal for charge balancing and to provide the compensation current signal to the output of the stimulation signal generating unit for compensating an unbalanced charge of the stimulation current signal and forming a compensated stimulation signal at the output of the stimulation signal generating unit.

A mismatch between positive and negative parts of a waveform may cause an unbalanced charge to electrodes connected to the stimulator circuit. The unbalanced charge may introduce an offset voltage build-up onto the electrodes over time, in particular as the interface between the electrode and tissue is often very capacitive. If the stimulator device is used to continuously output a stimulation signal, such as a burst of the stimulation signal, for a long period of time, the unbalanced charge can cause safety issues to the electrode and/or the tissue. For instance, the offset voltage build-up may form such a large electrical field that electrical breakdown of water occurs. The offset voltage build-up may also cause the electrode to break such that a subject to the stimulation may be exposed to toxic substances.

The compensation unit may be configured to provide the compensation current signal during output of the stimulation current signal. Thanks to the compensation current signal provided by the compensation unit, any unbalanced charge may thus be compensated during output of the stimulation signal, such as during a burst of the stimulation signal. Thus, the stimulator device may allow the burst to be output continuously for a long period of time without any need to interrupt stimulation in order for charge balance compensation to be performed.

By using the compensation unit, the stimulator device may ensure that charge balancing is provided so as to compensate for any mismatch between positive and negative parts of the waveform of the stimulation signal. This implies that the stimulator device may provide compensation instead of (or in combination with) control of functionality of components of the stimulator device.

The stimulation signal generating unit may comprise components having nonideal function which may cause an unbalance in the waveform. If the stimulation signal generating unit is to be provided in an integrated circuit, compensation of nonideal function of components may be difficult or impossible to achieve in the integrated circuit. Thanks to providing the compensation unit, the stimulator device may still ensure that an unbalanced charge is compensated to improve safety of the stimulator circuit. Hence, the stimulation signal generating unit may be provided in an integrated circuit providing a very small form factor of the stimulation signal generating unit.

The stimulator device may be particularly useful for generating stimulation signals that are to be used for temporal interference stimulation, wherein multiple stimulation signals interfere for causing an interferential signal within a brain and/or a nerve to be stimulated, such that the interferential signal may provide a desired stimulation.

In temporal interference stimulation, stimulation may be provided continuously over relatively long time and/or with a large number of bursts of individual stimulation signals. In order to avoid crosstalk between different stimulation signals, independent current source and current sink may be used for each stimulator device. This implies that there is a high risk of mismatch between the current source and the current sink such that build-up of an offset voltage onto electrodes may be likely. Thus, use of the compensation unit may ensure that offset voltage build-up in temporal interference stimulation is avoided.

The stimulation signal is provided at the output of the stimulation signal generating unit. Based on the stimulation signal, the compensation current signal is generated and also provided at the output of the stimulation signal generating unit. Thus, the compensation current signal is combined with the stimulation signal at the output to define the compensated stimulation signal. Hence, forming of the compensated stimulation signal may involve combining the stimulation signal at the output of the stimulation signal generating unit with the compensation current signal.

According to an embodiment, the compensation unit comprises an extreme voltage detector for detecting the extreme voltage of the voltage signal, wherein the impedance monitoring unit is configured to receive the detected extreme voltage from the compensation unit for determining the extreme voltage.

The compensation unit may be configured to determine the compensation current signal based on a common mode voltage of the stimulation signal. Thus, the compensation unit may be configured to detect the common mode voltage in order to detect presence of an unbalanced charge, which may need to be compensated for by the compensation unit.

Thus, the compensation unit may be configured to perform extreme point detection of the stimulation signal for determining an unbalance of the common mode voltage based on the stimulation signal. This implies that the compensation unit may be configured to detect the extreme voltage, which is also needed by the impedance monitoring unit. Hence, the extreme voltage can be used both by the compensation unit and the impedance monitoring unit and needs only be detected by one of the units.

It should be realized that if the stimulation current signal generated by the stimulation signal generating unit has a relatively short duration, no compensation unit may be needed.

According to an embodiment, the impedance monitoring unit comprises a counter for determining a time delay representative of the phase delay.

The counter may be configured to determine a time delay between corresponding points on the stimulation current signal and the voltage signal at the output. The counter may thus determine the time delay between corresponding points on waveforms. Using knowledge of frequency of the stimulation current signal, the time delay may be converted to a phase. This conversion need not necessarily be performed by the impedance monitoring unit. Rather, the impedance monitoring unit may be configured to output the time delay as a representation of the phase delay.

The counter may be configured to receive a trigger signal from the stimulation signal generating unit. For instance, the stimulation signal generating unit may be configured to generate the stimulation current signal in a controlled manner, such that a trigger may be provided to the counter corresponding to an extreme point, such as a peak, of the stimulation current signal. The counter may thus start counting based on receipt of the trigger.

The counter may further receive a trigger to stop counting based on detection of the extreme voltage of the voltage signal. Thus, the counter may be configured to determine the time delay based on determining a time period between the extreme point of the stimulation current signal and the following extreme point of the voltage signal.

According to an embodiment, the impedance monitoring unit being configured to determine the extreme voltage comprises the impedance monitoring unit being configured to determine both an upper extreme voltage at an upper extreme point of the voltage signal and a lower extreme voltage at a lower extreme point of the voltage signal.

This may be used for improving resolution of determining the impedance based on the information acquired by the impedance monitoring unit.

The upper extreme voltage and the lower extreme voltage may be used together with a phase delay associated with each of the upper extreme voltage and the lower extreme voltage for determining the impedance of the interface between the electrode and tissue.

The upper extreme point corresponds to a local maximum of the voltage signal. The upper extreme voltage is a value of the voltage signal at the upper extreme point. The lower extreme point corresponds to a local minimum of the voltage signal. The lower extreme voltage is a value of the voltage signal at the lower extreme point.

The processor may be configured to receive the upper extreme voltage and the lower extreme voltage from the impedance monitoring unit. The processor may further be configured to receive phase delays associated with each of the upper extreme voltage and the lower extreme voltage. The processor may be configured to use this information for determining the impedance with a high resolution.

According to an embodiment, the stimulation current signal comprises a sequence of temporally separated bursts, wherein each burst comprises an alternating current (AC) signal having the sinusoidal-like waveform.

The stimulation current signal comprising bursts may be suitable for use in temporal interference stimulation, wherein multiple bursts in a sequence may provide a desired stimulating effect in tissue, such as in a brain and/or a nerve. The use of bursts imply that power consumption may be limited since the stimulation current signal need not be constantly output over a long period of time. In addition, the use of bursts also allows compensation for unbalanced charges that may build up over a continuous output of a stimulation current signal, such that safety issues related to build-up of charges at the interface between the electrode and tissue may be avoided.

The impedance monitoring unit is configured to determine the extreme voltage of the voltage signal and the phase delay, while a burst of the stimulation current signal is provided on the output.

According to an embodiment, the stimulator device is configured to, between bursts of the stimulation current signal, reset a direct current (DC) voltage at the output of the stimulation signal generating unit to a reference voltage.

After a burst of the stimulation current signal, a remaining offset voltage may be present at the output of the stimulation signal generating unit. The remaining offset voltage may be present even if a compensation unit is used for compensating unbalanced charges during output of a burst.

Thanks to the stimulator device being configured to reset the DC voltage at the output, safety issues related to build-up of charges at the interface between the electrode and tissue may be avoided.

According to an alternative, the remaining offset voltage may be compensated for by controlling a reference voltage being used by the compensation unit for determining the compensation current signal. The reference voltage may be adjusted based on the remaining offset voltage after a burst such that the compensation current signal generated by the compensation unit during a next burst will compensate for the remaining offset voltage.

According to an embodiment, the stimulator device further comprises a power management unit configured to control a supply voltage controlling generation of the stimulation current signal by the stimulation signal generating unit based on the determined extreme voltage.

The supply voltage may define a maximum available voltage level of the voltage signal formed at the output of the stimulation signal generating unit. However, the voltage level of the voltage signal may depend on an amplitude to be used by the stimulation signal and on impedance of the interface between the electrode and tissue. Thus, if no power management unit is used, the supply voltage should be sufficient to support a worst-case scenario of largest amplitude of the stimulation current signal and largest impedance.

The power management unit may be configured to set a supply voltage that supports the determined extreme voltage, possibly with a small margin. Thus, the supply voltage may be controlled such that power consumption is saved by avoiding use of an unnecessarily high supply voltage.

The extreme voltage may be determined for a burst in the stimulation signal. The power management unit may then control the supply voltage that is used for the next burst.

The power management unit may also be configured to control a reference voltage. The reference voltage may provide a DC voltage level, such that the stimulation current signal may apply the sinusoidal-like waveform superposed on the DC voltage level. The reference voltage may thus be used for resetting a voltage at the output of the stimulation signal generating unit to the reference voltage.

According to a second aspect, there is provided a system for providing temporal interference stimulation of a body part of a living being, said system comprising: a first pair of electrodes configured to be arranged in a first relation to the body part; a second pair of electrodes configured to be arranged in a second relation to the body part; wherein each electrode of the first pair of electrodes and the second pair of electrodes forms part of a respective stimulator device according to the first aspect.

Effects and features of this second aspect are largely analogous to those described above in connection with the first aspect. Embodiments mentioned in relation to the first aspect are largely compatible with the second aspect.

Thus, a plurality of stimulator devices may be part of a system for providing stimulation of a body part of a living being, such as a human being or an animal. The body part may for instance be a brain and/or a nerve of the living being, but it should be realized that it may also or alternatively be a spinal cord, or a dorsal route ganglion. Each stimulator device may be used for providing a stimulation current signal at a respective electrode.

The system is able to monitor impedance of the electrode-tissue interface for each of the electrodes. This may provide clinical information relating to the electrode-tissue interface. In addition, the system may use the impedance information for saving power in generating of the stimulation current signals to be provided to the respective electrodes.

A first and a second pair of stimulator devices may be used for output of stimulation signals to a first and a second pair of electrodes. A first stimulator device may thus be combined with a second stimulator device to form the first pair of stimulator devices and a third stimulator device may be combined with a fourth stimulator device to form the second pair of stimulator devices. The first stimulator device and the second stimulator device may be configured to receive an identical current waveform, which may be copied and optionally amplified by a current source and a current sink. The first stimulator device and the second stimulator device may be controlled such that the first stimulator device may enable the current source when the second stimulator device enables the current sink, and vice versa. Each of the first stimulator device and the second stimulator device may be connected to a respective electrode of the first pair of electrodes which may thus receive stimulation current signals that are in anti-phase for forming a stimulation signal to be transmitted into the body part, such as the brain and/or the nerve.

The third stimulator device and the fourth stimulator device may generate stimulation current signals in the same way as described above for the first stimulator device and the second stimulator device. Each of the third stimulator device and the fourth stimulator device may be connected to a respective electrode of the second pair of electrodes which may thus receive stimulation current signals that are in anti-phase for forming a stimulation signal to be transmitted into the body part, such as the brain and/or the nerve.

It should further be realized that the system may comprise a set of electrodes, such that the electrode to be used for transmitting of the stimulation current signal into the body part, such as the brain and/or nerve, may be dynamically selected for each stimulator device when the system is used.

It should be realized that the system may comprise more than two pairs of stimulator devices for forming more than two stimulation signals in the tissue. A number of stimulator devices of the system may be dependent on the stimulation to be provided. For instance, in some embodiments, the system may comprise more than two pairs of stimulator devices for generating more than two stimulation signals for causing an interferential stimulation signal to be formed based on more than two stimulation signals.

According to an embodiment, the system further comprises a reference electrode for defining a reference voltage in relation to each electrode of the first pair of electrodes and the second pair of electrodes.

Thus, a separate reference voltage may be provided to the tissue. The reference electrode provides a bias voltage to tissue.

According to an embodiment, the system comprises a common power management unit for controlling a common supply voltage of the stimulation signal generating units of each of the stimulator devices.

Thus, the stimulation signal generating units may receive a common supply voltage. This implies that a single supply voltage may be controlled for providing power saving in the system.

According to an alternative embodiment, a respective supply voltage controlling generation of the stimulation current signal by the respective stimulation signal generating units is individually controlled.

Thus, each stimulation signal generating unit of each of the stimulator devices in the system may receive an individually controlled supply voltage. Thus, different supply voltages may be provided to different stimulation signal generating units. This implies that power may be saved individually for each of the stimulation signal generating units, such that more power may be saved compared to using a common supply voltage.

The supply voltages for each of the stimulation signal generating units may be determined by a common power management unit. Thus, a single power management unit may be used, which receives input for determining the supply voltages for each of the stimulation signal generating units. The single power management unit may thus provide a plurality of output signals, each output signal being dedicated for a unique stimulation signal generating unit.

The power management unit may also be configured to control a reference voltage. The reference voltage may provide a DC voltage level, such that the stimulation current signal may apply the sinusoidal-like waveform superposed on the DC voltage level. The reference voltage may thus be used for resetting a voltage at the output of the stimulation signal generating unit to the reference voltage.

The reference voltage may also be applied to the reference electrode. Since all of the electrodes of the system may be related to the common reference voltage, the power management unit may control a single reference voltage which may be used by each of the stimulator devices.

According to a third aspect, there is provided a method for monitoring a stimulation signal, said method comprising: generating a stimulation current signal and providing the stimulation current signal to an output of a stimulation signal generating unit, wherein the stimulation current signal comprises a sinusoidal-like waveform; while the stimulation current signal is provided on the output of the stimulation signal generating unit, determining an extreme voltage at an extreme point of a voltage signal at the output and a phase delay between the voltage signal and the stimulation current signal for determining information of an impedance relating to an interface between an electrode, connected to the output, and tissue.

In particular, the method may be performed in the stimulator device of the first aspect or in the system according to the second aspect.

Effects and features of this third aspect are largely analogous to those described above in connection with the first and second aspects. Embodiments mentioned in relation to the first and second aspects are largely compatible with the third aspect.

The method is able to monitor impedance of the electrode-tissue interface for an electrode, while a stimulation current signal is output. This may provide clinical information relating to the electrode-tissue interface. In addition, the impedance information may be used for saving power in generating of the stimulation current signals to be provided at the output.

According to a fourth aspect, there is provided a stimulator system for providing temporal interference stimulation of a body part of a living being, said stimulator system comprising: a first stimulator module configured to generate a first stimulation current signal and provide the first stimulation current signal to an output of the first stimulator module, wherein the first stimulation current signal comprises a first burst comprising a sinusoidal-like waveform; a second stimulator module configured to generate a second stimulation current signal and provide the second stimulation current signal to an output of the second stimulator module, wherein the second stimulation current signal comprises a second burst comprising a sinusoidal-like waveform; a first pair of electrodes configured to be arranged in a first relation to the body part and configured to receive the first stimulation current signal; and a second pair of electrodes configured to be arranged in a second relation to the body part and configured to receive the second stimulation current signal; wherein the first stimulator module and the second stimulator module are configured to generate and output the first stimulation current signal and the second stimulation current signal with a difference in frequency of the first burst and the second burst, wherein the second stimulator module is further configured to be controlled for providing a varying amplitude and/or frequency within the second burst; and wherein the stimulator system is configured to stimulate the body part using interferential stimulation based on interference between the first stimulation current signal and the second stimulation current signal.

The stimulator system according to the fourth aspect is configured to generate stimulation current signals which may be used for generating an interferential signal within a body part of the living being, such as a human being or an animal. The body part may for instance be a brain and/or a nerve of the living being, but it should be realized that it may also or alternatively be a spinal cord, or a dorsal route ganglion. The stimulator system provides detailed control of the interferential signal by controlling the amplitude and/or frequency within the second burst provided by the second stimulation generating unit.

This implies that the interferential signal formed by interference of the first stimulation current signal and the second stimulation current signal may be accurately controlled. In particular, variation of the amplitude and/or frequency of the second burst can be used for forming a gradually changing interferential signal. In addition, phase of the second stimulation current signal in the second burst in relation to phase of the first stimulation current signal may be used for controlling the interferential signal. This may be particularly advantageous for using the interferential signal for blocking propagation of a neural signal in the brain and/or nerve. However, it should be realized that the control of the amplitude and/or frequency of the second burst may be used for controlling the waveform of the interferential signal which may alternatively or additionally be used for other purposes of stimulating the brain and/or the nerve.

It should be realized the stimulator system of the fourth aspect may be used in combination with the features according to any of the embodiments mentioned in relation to the first, second, and third aspects. In addition, embodiments mentioned in relation to the fourth aspect are largely compatible with the first, second, and third aspects.

The sinusoidal-like waveform of the first burst may have a first frequency. The sinusoidal-like waveform of the second burst may have a second frequency, different from the first frequency.

As mentioned above, temporal interference stimulation corresponds to two or more stimulation signals interfering for causing an interferential signal in the body part to be stimulated. The interferential signal may be controlled for providing a desired stimulation.

When the first stimulation current signal and the second stimulation current signal interfere, the interference may form a temporally varying signal having a frequency corresponding to the difference in frequency between the first burst and the second burst, i.e., a difference between the first frequency and the second frequency. This frequency of the interference between the first and second stimulation current signals may be referred to as a beat frequency.

The first stimulation current signal is configured to be provided to a first pair of electrodes arranged in a first relation to the body part, such as the brain and/or the nerve. This implies that the first stimulation current signal will form an electric field in the body part, such as the brain and/or the nerve, at the location of the first pair of electrodes. Further, the second stimulation current signal is configured to be provided to a second pair of electrodes arranged in a second relation to the body part, such as the brain and/or the nerve. This implies that the second stimulation current signal will form an electric field in the body part, such as the brain and/or the nerve, at the location of the second pair of electrodes. These electric fields may interfere within the body part, such as the brain and/or the nerve, for forming the interferential stimulation.

According to an embodiment, each electrode of the first pair and the second pair may be associated with a respective stimulator device. Thus, each stimulator module may be formed by two stimulator devices as defined in the first aspect above. The two stimulator devices may be configured to output stimulation current signals that are in anti-phase for forming the stimulation current signals.

Each stimulator device of the first stimulator module may be connected to a respective electrode of the first pair of electrodes which may thus receive stimulation current signals that are in anti-phase for forming the first stimulation current signal to be transmitted into the body part, such as the brain and/or the nerve. Similarly, each stimulator device of the second stimulator module may be connected to a respective electrode of the second pair of electrodes which may thus receive stimulation current signals that are in anti-phase for forming the second stimulation current signal to be transmitted into the body part, such as the brain and/or the nerve.

Each stimulator device may comprise a current source and a current sink. The current source and the current sink may thus form independent components for generating a stimulation current signal at an output node. The use of independent components may avoid crosstalk between the stimulator devices during output of a stimulation signal at the output node.

The stimulator devices of a stimulator module may be configured to receive an identical current waveform, which may be copied and optionally amplified by a current source and a current sink. The stimulator devices may be controlled such that one stimulator device may enable the current source when the other stimulator device enables the current sink, and vice versa. This may ensure that the stimulator devices of the stimulator module provides signals that are in anti-phase, which are then received by a respective electrode in a pair of electrodes.

According to an embodiment, the electrodes within the first pair of electrodes may be slightly displaced in relation to each other along an extension of the body part, such as the brain and/or the nerve, along which extension signals are propagated in the body part, such as the brain and/or the nerve. Similarly, the electrodes within the second pair of electrodes may be slightly displaced in relation to each other along the extension of the body part, such as the brain and/or the nerve.

The electrodes of the first and second pairs may be arranged at corresponding cross-sections of the body part, such as the brain and/or the nerve, such that a first electrode of the first pair and a first electrode of the second pair are arranged in relation to a same first cross-section of the body part, such as the brain and/or the nerve, whereas a second electrode of the first pair and a second electrode of the second pair are arranged in relation to a same second cross-section of the body part, such as the brain and/or the nerve.

The first stimulation current signal and the second stimulation current signal may thus each define electric fields along a common extension of the body part, such as the brain and/or the nerve, with the electric fields extending into the body part, such as the brain and/or the nerve, so as to form interferential stimulation.

According to another embodiment, the first pair of electrodes and the second pair of electrodes are arranged at different locations of a circumference of the nerve. Thus, the first stimulation current signal and the second stimulation current signal may mainly define electric fields based on the signal in different portions of a cross-section of the nerve. The first stimulation current signal and the second stimulation current signal may interfere within the nerve to stimulate the nerve through interferential stimulation.

Both electrodes of the first pair of electrodes may be associated with a first part of the circumference of the nerve and both electrodes of the second pair of electrodes may be associated with a second part of the circumference of the nerve, wherein the second part is different from and non-overlapping with the first part. This implies that the portions of the cross-section of the nerve in which the first stimulation current signal and the second stimulation current signal, respectively, define electric fields are quite different so as to allow interferential stimulation deep within the nerve without necessarily providing stimulation in undesired locations of the nerve.

Alternatively, the electrodes of the first pair of electrodes and the electrodes of the second pair of electrodes may be alternatingly arranged around the circumference of the nerve, such that an electrode of the first pair of electrodes is arranged between the electrodes of the second pair of electrodes along the circumference of the nerve. This implies that the first stimulation current signal and the second stimulation current signal may define electric fields through the nerve with a large area of overlap of the electric fields based on the first stimulation current signal and the second stimulation current signal, respectively.

All of the electrodes of the first pair of electrodes and the second pair of electrodes may be provided in relation to a common cross-section of the nerve.

It should further be realized that the system may comprise a set of electrodes, such that the electrode to be used for transmitting of the stimulation current signal into the body part, such as the brain and/or nerve, may be dynamically selected for each stimulator device when the system is used. Thus, the first pair of electrodes and the second pair of electrodes may be dynamically defined when the first stimulation current signal and the second stimulation current signal is to be output.

It should further be realized that more than two pairs of electrodes may be used. Thus, additional stimulation current signals may be output to additional pairs of electrodes for forming the interferential stimulation on the additional stimulation current signal in addition to the interference between the first and second stimulation current signals.

The first frequency and the second frequency may be selected to be high frequencies, whereas the beat frequency represents a low frequency lower than the first frequency and the second frequency. The body part, such as the brain and/or the nerve, may be affected to a larger extent by low frequency signals than high frequency signals. Thus, the first stimulation current signal and the second stimulation current signal may include a first frequency and a second frequency, respectively.

Further, the first stimulation current signal and the second stimulation current signal may be configured to interfere, such as constructively or destructively interfere in the body part, such as the brain and/or the nerve, for forming the interferential signal. Thus, in some cases, an amplitude of the first stimulation current signal and an amplitude of the second stimulation current signal may be smaller than an amplitude of the interferential signal with the beat frequency, such that a sufficiently large signal (amplitude) for stimulating the nerve is only provided by the interferential signal.

The interferential stimulation may be controlled by controlling the first stimulation current signal and/or the second stimulation current signal. This may imply that a location within the body part, such as the brain and/or the nerve, in which stimulation is provided may be controlled. The control of the interferential stimulation may be provided by controlling one or more of a frequency of the stimulation current signal, an amplitude of the stimulation current signal, or a location of electrodes of the pairs of electrodes to which the stimulation current signals are output.

Thus, an overall control of the interferential stimulation may be provided. This may be used for controlling a location within the body part, such as the brain and/or the nerve, to be stimulated. In addition or alternatively to the overall control, the second stimulator module may be controlled for providing a varying amplitude and/or frequency within the second burst. The varying amplitude and/or frequency provides a gradually changing interferential signal, forming a time-varying interferential signal.

The system may thus be configured to form a gradual time-varying interferential signal in the body part, such as the brain and/or the nerve. The time-varying interferential signal may be controlled such that the interferential signal do not include a step function of large changes in the interferential signal. This implies that stimulation for triggering a neural signal to be generated in the body part, such as the brain and/or the nerve, may be avoided. However, the time-varying interferential signal is configured to affect an activation threshold in the body part, such as the brain and/or the nerve, for allowing propagation of neural signals. Thus, by the time-varying interferential signal being configured to increase the activation threshold, the interferential signal may prevent propagation of neural signals in the body part, such as the brain and/or the nerve, without triggering generation of neural signals.

The system may thus be configured to locally selectively block propagation of neural signals. Propagation of neural signals may be blocked in a selected location in the body part, such as the brain and/or the nerve, wherein the interferential signal is controlled by the system for providing blocking in the selected location while not necessarily blocking other signals propagating in the body part, such as the brain and/or the nerve. Thus, propagation of signals may be selectively blocked for one or more nerve fibers among a plurality of nerve fibers in the body part, such as the brain and/or the nerve.

The system may further be configured to temporally selectively block propagation of neural signals. Propagation of neural signals may be blocked at a time when the first and second bursts are output.

It should be realized that, even though control of the second stimulator module for providing a varying amplitude and/or frequency is described above, the first stimulator module may also be controlled. Thus, the first stimulator module may be configured to be controlled for providing a varying amplitude and/or frequency within the first burst. The system may be configured to control the first stimulator module and the second stimulator module in order for the control of the first burst and the second burst causing a desired time-varying interferential signal to be formed.

As mentioned above, the system may be used for temporally and locally selective blocking of propagation of signals. This may be useful for preventing propagation of undesired signals in the body part, such as the brain and/or the nerve. The system may thus be used for pain management, wherein blocking of propagation of pain signals in the body part, such as the brain and/or the nerve, may be used for pain relief of the living being.

The system may be configured to modulate particular nerves involved in causing pain rather than on systemic medication that can have widespread effects on the living being. This targeted approach reduces the risk of systemic side effects and dependency associated with medication for pain relief. Thus, the system may be particularly useful for patients with chronic pain conditions.

Additionally, the system allows for adjustable and reversible pain control. Stimulation parameters can be tailored to a pattern of pain signals transmitted in the living being, offering personalized therapy. Furthermore, the system can be adjusted or turned off if needed, providing flexibility in managing pain levels.

The system may be efficiently used, e.g., for individuals with neuropathic pain, failed back surgery syndrome, and other chronic pain conditions that have been difficult to manage using traditional approaches.

It should be realized that even though the use of the system for blocking propagation of neural signals is discussed above, the system may be used for other types of stimulation of the body part, such as the brain and/or the nerve. As explained above, the time-varying interferential signal may be particularly advantageous for providing blocking of propagation of neural signals without causing triggering of any neural signals. However, the control of at least the second stimulator module causing a time-varying interferential signal may be used for controlling a waveform of the time-varying interferential signal to be adapted to a desired effect. Thanks to the interferential signal having a time-varying waveform, a flexibility in the waveform generated is provided, which may be utilized in various applications for stimulating the body part, such as the brain and/or the nerve.

The first stimulation current signal may comprise a sequence of first bursts. Each first burst in the sequence may comprise a sinusoidal-like waveform defining a first frequency. The second stimulation current signal may comprise a sequence of second bursts. Each second burst in the sequence may comprise a sinusoidal-like waveform defining a second frequency.

Thus, the interferential signal may be formed for affecting the body part, such as the brain and/or the nerve, in a plurality of time points corresponding to time points of the bursts. The bursts may be output periodically at regular time intervals or intermittently, possibly with separate control of each time a burst is to be output.

According to an embodiment, the system further comprises a sensor unit for detecting propagation of a neural signal, wherein the first stimulator module and the second stimulator module are configured to output the first stimulation current signal and the second stimulation current signal for blocking propagation of the neural signal.

Thus, the system allows detecting a neural signal to be blocked by the interferential signal. Measurements by the sensor unit may be provided to a control unit of the stimulator module and, when a neural signal to be blocked is detected, the control unit may trigger output of the first burst of the first stimulation current signal and the second burst of the second stimulation current signal for forming the interferential signal for blocking propagation of the neural signal.

This implies that the system may be configured to only provide blocking of propagation of signals when an undesired neural signal is passing a location at which temporal interference stimulation is provided.

The sensor unit may comprise any sensor suitable for detecting propagation of a neural signal. For instance, the sensor unit may comprise one or more pairs of electrodes being configured to be arranged in relation to the body part, such as the brain and/or the nerve. The electrodes may be configured to sense an electrical signal propagating in the body part, such as the brain and/or the nerve, for sensing the neural signal. For instance, the electrodes of the sensor unit may be configured to be arranged in contact with the body part, such as the brain and/or the nerve, such as in contact with a surface of the body part, such as the brain and/or the nerve.

The sensor unit may be configured to be arranged in relation to a first longitudinal position of the body part, such as the brain and/or the nerve. The first pair of electrodes and the second pair of electrodes of the system for output of the first stimulation current signal and the second stimulation current signal, respectively, may be configured to be arranged in relation to a second longitudinal position of the body part, such as the brain and/or the nerve.

The body part, such as the brain and/or the nerve, may have a longitudinal extension along which neural signals propagate. It should be realized that the first longitudinal position and the second longitudinal position refer to separate positions along the longitudinal extension of the body part, such as the brain and/or the nerve.

The second longitudinal position may be arranged in relation to the first longitudinal position such that a neural signal to be blocked passes the first longitudinal position before reaching the second longitudinal position.

The sensor unit may be configured to be arranged in a common carrier with the stimulator module and the first and second pair of electrodes for providing stimulation signals. Thus, the carrier may have a longitudinal extension for extending along the body part, such as the brain and/or the nerve. Alternatively, the sensor unit may be arranged in a separate carrier.

The sensor unit may be connected by a wired connection to the control unit for providing measurement results to the control unit.

Timing of output of the first and second bursts may be the controlled such that an activation threshold is increased shortly before the neural signal to be blocked reaches the second longitudinal position. The control unit may be configured to control output based on a knowledge of speed of the neural signal and a knowledge of distance between the first and second longitudinal positions. The timing may also or alternatively be controlled based on feedback and/or calibration of the system.

The sensor unit may comprise a plurality of individual sensors, which may be arranged at different longitudinal positions in relation to the body part, such as the brain and/or the nerve.

The plurality of individual sensors may be used for detecting propagation of the neural signal through the different longitudinal positions. This implies that a speed of the neural signal may be determined based on a time delay of detecting the neural signal at the different longitudinal positions. Information of the speed of the neural signal may further be used by the control unit for timing control of output of the first and second bursts.

According to an embodiment, the stimulator system may further comprise a feedback sensor unit for detecting propagation of the neural signal.

The feedback sensor unit may comprise any sensor suitable for detecting propagation of a neural signal. For instance, the feedback sensor unit may comprise one or more pairs of electrodes being configured to be arranged in relation to the body part, such as the brain and/or the nerve. The electrodes may be configured to sense an electrical signal propagating in the body part, such as the brain and/or the nerve, for sensing the neural signal. For instance, the electrodes of the feedback sensor unit may be configured to be arranged in contact with the body part, such as the brain and/or the nerve, such as in contact with a surface of the body part, such as the brain and/or the nerve.

The feedback sensor unit may be configured to be arranged in relation to a third longitudinal position of the body part, such as the brain and/or the nerve. The third longitudinal position may be arranged in relation to the first and second longitudinal position such that a neural signal travels from the first longitudinal position to the second longitudinal position and then further to the third longitudinal position. Thus, the sensor unit and the feedback sensor unit may be configured to be arranged at opposite sides of the first and second pairs of electrodes of the system for output of the first stimulation current signal and the second stimulation current signal, respectively.

The feedback sensor unit may be configured to sense whether a neural signal to be blocked has been successfully blocked. Thus, the feedback sensor unit may be configured to provide feedback for controlling the output of the first and second stimulation current signals. For instance, a timing, an amplitude and/or a frequency of the first and second bursts of the first and second stimulation current signals may be adapted for controlling the system to efficiently block an undesired neural signal. Also or alternatively, electrodes selected for output of the first and second stimulation current signals may be adapted for controlling the system to efficiently block an undesired neural signal.

According to an embodiment, the second stimulation signal is configured to be controlled for decreasing the difference in frequency of the first burst and the second burst during the first burst and the second burst.

This may be particularly suitable for ensuring that the interferential signal may provide blocking of propagation of neural signals, while not triggering generation of neural signals.

During output of the first burst and the second burst, the interferential signal may thus have a beat frequency which is initially relatively high and then gradually decreases. The body part, such as the brain and/or the nerve, may be less likely to be triggered to generate a neural signal when stimulated by a high frequency signal compared to when stimulated by a low frequency signal. Thus, thanks to the interferential signal having a decreasing beat frequency, the body part, such as the brain and/or the nerve, will not be triggered to generate a neural signal, while the interferential signal may cause a change of the activation threshold for providing blocking of propagation of neural signals.

It should be realized that the first burst may have a first frequency which is constant and relatively high. The second stimulator module may thus be configured to be controlled for providing a second burst with a second frequency which is gradually increasing during the second burst, while the second frequency is held lower than the first frequency. Thus, the second frequency may initially be lower than the first frequency and may be changed during the second burst to remain lower than the first frequency but being closer to the first frequency.

It should be further realized that, in some embodiments, the second stimulation signal is configured to be controlled for increasing the difference in frequency of the first burst and the second burst during the first burst and the second burst. During output of the first burst and the second burst, the interferential signal may thus have a beat frequency which gradually increases. This may be useful for blocking or promoting propagation of neural signals.

According to an embodiment, the second stimulator module is further configured to be controlled for providing a plurality of consecutive stimulation cycles within the second burst, wherein the second stimulation current signal during each stimulation cycle comprises an integer number of sinusoidal waves with a constant amplitude and frequency.

This implies that each stimulation cycle is configured to provide symmetrical positive and negative parts of a waveform during each stimulation cycle. This implies that a charge balanced stimulation may be provided during each stimulation cycle.

However, it should be realized that the stimulator module may still not provide a perfect charge balance of the first and second stimulation current signals. Thus, a mismatch between positive and negative parts of a waveform may exist which may cause an unbalanced charge to electrodes connected to the stimulator modules. Thus, as discussed above in an embodiment of the first aspect, the stimulator system of the fourth aspect may comprise a compensation unit for charge balancing.

According to an embodiment, the stimulator system further comprises a first compensation unit configured to generate a first compensation current signal for charge balancing of the first stimulation current signal and to provide the first compensation current signal to the output of the first stimulator module for compensating an unbalanced charge of the first stimulation current signal and forming a compensated first stimulation current signal at the output of the first stimulator module; and a second compensation unit configured to generate a second compensation current signal for charge balancing of the second stimulation current signal and to provide the second compensation current signal to the output of the second stimulator module for compensating an unbalanced charge of the second stimulation current signal and forming a compensated second stimulation current signal at the output of the second stimulator module.

Each of the first and the second compensation unit may function in a same manner. For brevity, only the first compensation unit is discussed below but it should be realized that the discussion may equally apply to the second compensation unit.

The first compensation unit may be configured to provide the first compensation current signal during output of the first stimulation current signal. Thanks to the first compensation current signal provided by the first compensation unit, any unbalanced charge may thus be compensated during output of the first stimulation current signal, such as during the first burst(s) of the first stimulation current signal.

By using the compensation units, the stimulator system may ensure that charge balancing is provided so as to compensate for any mismatch between positive and negative parts of the waveform of the stimulation current signals. This implies that the stimulator system may provide compensation instead of (or in combination with) control of functionality of components of the stimulator system.

The second compensation unit may be configured to detect an unbalanced charge for each stimulation cycle within the second burst and may further be configured to output the second compensation current signal during a following stimulation cycle. Similarly, the first compensation unit may be configured to detect an unbalanced charge for an integer number of sinusoidal waves and may provide the first compensation current signal for following sinusoidal waves.

The stimulator system may thus be configured to provide active charge balancing during output of the first and second stimulation current signals. Even though active charge balancing is provided, a residue charge may still remain after output of the first and second bursts.

The stimulator system may further comprise a first switch for shorting the output of the first stimulator module and a second switch for shorting the output of the second stimulator module.

The outputs of the first and second stimulator module, respectively, may be shorted to a known common mode potential for discharging any residue charge at the electrodes. This may ensure absolute charge balancing of the stimulator system.

According to an embodiment, the first stimulator module is configured to, between first bursts of the first stimulation current signal, reset a DC voltage at the output of the first stimulator module to a reference voltage and the second stimulator module is configured to, between second bursts of the second stimulation current signal, reset a DC voltage at the output of the second stimulator module to a reference voltage.

Each of the first and the second stimulator modules may function in a same manner for resetting DC voltages. For brevity, only the first stimulator module is discussed below but it should be realized that the discussion may equally apply to the second stimulator module.

After a first burst of the first stimulation current signal, a remaining offset voltage may be present at the output of the first stimulator module. The remaining offset voltage may be present even if the first compensation unit is used for compensating unbalanced charges during output of the first burst.

Thanks to the first stimulator module being configured to reset the DC voltage at the output, safety issues related to build-up of charges at the interface between the electrode and tissue may be avoided.

According to an alternative, the remaining offset voltage may be compensated for by controlling a reference voltage being used by the first compensation unit for determining the first compensation current signal. The reference voltage may be adjusted based on the remaining offset voltage after a first burst such that the first compensation current signal generated by the first compensation unit during a next burst will compensate for the remaining offset voltage.

According to an embodiment, the stimulator system comprises a memory for storing parameters for forming a waveform of the first burst and a waveform of the second burst.

The parameters may be determined in a separate processing unit and may be provided to the stimulator system. For instance, the stimulator system may be configured to be implanted in the living being. Thus, the stimulator system may have limited processing resources and the processing for determining the parameters for generating the first and second stimulation current signals may preferably be performed in an external unit.

The stimulator system may be configured to receive updated parameters for changing the first and second stimulation current signals. Alternatively, the stimulator system may be configured with static parameters for always providing the same first and second stimulation current signals.

Also, it should be realized that the stimulator system may be configured to determine adjustments of the parameters for adjusting the first and second stimulation current signals based on any feedback, such as feedback received from the feedback sensor unit.

The parameters may include a sequence of stimulation direction values (one or zero representing positive/negative value of the stimulation current signal) and digital code values representing an absolute value of the stimulation current signal. The stimulation direction values and digital code values may be associated with respective time points.

The memory may store complete parameters for generating the first and second bursts. For instance, the memory may store individual parameters for generating each of the stimulation cycles within a second burst. This may imply that limited processing is required by the stimulator system for generating the first and second bursts. However, a relatively large memory may be needed for storing the parameters.

According to an embodiment, the memory may store parameters for a single sinusoidal waveform and the stimulator system may be configured to generate the first burst and the second burst based on the parameters.

Thus, the stimulator system may be configured to generate a varying frequency in the first burst and/or the second burst based on the single sinusoidal waveform.

The memory may store digital code values representing an absolute value of the stimulation current signal for half a period of the single sinusoidal waveform.

The stimulator system may further store or be provided with adjustable parameters for generating sinusoidal waveforms with a varying frequency and/or amplitude. The adjustable parameters may include a stimulation direction, a value for controlling a peak amplitude, and a step size for controlling the frequency.

According to a fifth aspect, there is provided a method for providing temporal interference stimulation of a body part of a living being; said method comprising: generating a first stimulation current signal and providing the first stimulation current signal to an output connected to a first pair of electrodes arranged in a first relation to the body part, wherein the first stimulation current signal comprises a first burst comprising a sinusoidal-like waveform; generating a second stimulation current signal and providing the second stimulation current signal to an output connected to a second pair of electrodes arranged in a second relation to the body part, wherein the second stimulation current signal comprises a second burst comprising a sinusoidal-like waveform with a varying amplitude and/or frequency within the second burst; wherein the first stimulation current signal and the second stimulation current signal are generated and output to the first and second pairs of electrodes, respectively, with a difference in frequency of the first burst and the second burst; and wherein the body part is stimulated by interferential stimulation based on interference between the first stimulation current signal and the second stimulation current signal.

Effects and features of this fifth aspect are largely analogous to those described above in connection with the fourth aspect. Embodiments mentioned in relation to the fourth aspect are largely compatible with the fifth aspect.

It should be realized the method of the fifth aspect may be used in combination with the features according to any of the embodiments mentioned in relation to the first, second, and third aspects. In addition, embodiments mentioned in relation to the fifth aspect are largely compatible with the first, second, and third aspects.

The method allows accurate control of the interferential signal formed within the body part. The body part may for instance be a brain and/or a nerve of the living being, but it should be realized that it may also or alternatively be a spinal cord, or a dorsal route ganglion.

The method may be particularly useful for controlling blocking of propagation of neural signals.

### BRIEF DESCRIPTION OF THE DRAWINGS

The above, as well as additional objects, features, and advantages of the present description, will be better understood through the following illustrative and non-limiting detailed description, with reference to the appended drawings. In the drawings like reference numerals will be used for like elements unless stated otherwise.

FIG. 1 is a schematic view of a system according to a first embodiment.

FIG. 2 is a schematic view of stimulation current signals with power management of the system according to the first embodiment.

FIG. 3 is a schematic view of a system according to a second embodiment.

FIG. 4 is a schematic view of stimulation current signals with power management of the system according to the second embodiment.

FIG. 5 is a flow chart of a method according to an embodiment.

FIG. 6 is a schematic view of a stimulator system according to an embodiment.

FIG. 7 is a schematic view of an arrangement of the stimulator system in relation to a nerve according to an embodiment.

FIG. 8 is a schematic view of waveforms provided to electrodes of the stimulator system.

FIGS. 9a-b are schematic views of memory-based generation of stimulation current signals.

FIG. 10 is a flow chart of a method according to an embodiment.

### DETAILED DESCRIPTION

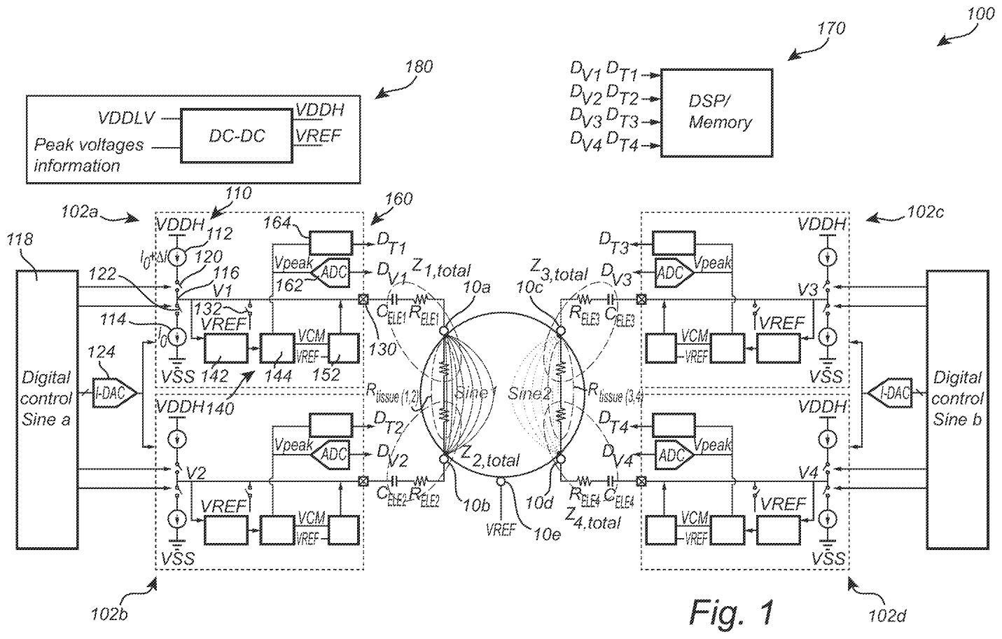

In FIG. 1, a system 100 for providing temporal interference stimulation of a brain and/or a nerve according to a first embodiment is shown. It should be realized that even though reference is made herein to the brain and/or the nerve being stimulated, the system 100 may also or alternatively be configured for stimulating a spinal cord, or a dorsal route ganglion. The system 100 comprises a plurality of stimulator devices 102a, 102b, 102c, 102d. Below, mainly a single stimulator device 110a will be described in detail for simplicity and brevity. In addition, it should be understood that the stimulator device 102a may be used in a different system. The stimulator device 102a may be used as a sole stimulator device 102a providing a stimulation signal. It should further be understood that each of the stimulator devices 102a, 102b, 102c, 102d may be implemented in a same manner, such that the description of the stimulator device 102a applies as well to the other stimulator devices 102b, 102c, 102d.

The stimulator device 102a comprises a stimulation generating unit 110 configured to generate a stimulation signal. The stimulation generating unit 110 may comprise a current source 112 and a current sink 114. The current source 112 may be connected between a supply voltage and an output node 116 and the current sink 114 may be connected between ground and the output node 116.

The stimulation generating unit 110 may further comprise a stimulation controller 118 which may provide digital control of a first switch 120 arranged between the current source 112 and the output node 116 and a second switch 122 arranged between the current sink 114 and the output node 116. The stimulation controller 118 may thus control which of the current source 112 and the current sink 114 is connected to the output node 116.

The current source 112 and the current sink 114 may thus form independent components for generating a stimulation current signal at the output node 116. The use of independent components may avoid crosstalk between the stimulator devices 102a, 120b, 102c, 102d, which may be used for example for providing interferential stimulation, during output of a stimulation signal at the output node 116.

The output node 116 may further be connected to an output 130 of the stimulation signal generating unit 110. The output 130 may further be connected, e.g., to an electrode 10 for providing the stimulation signal to a living being. It should be realized that the electrode 10a may not necessarily be part of the stimulator device 102a.

The stimulation signal generating unit 110 may be configured to provide the stimulation signal via the output node 116 to the output 130 of the stimulation signal generating unit 110. The stimulation signal may be a sinusoidal-like waveform, such as a pure sinusoidal waveform, a pseudo-sinusoidal waveform, a modified sinusoidal waveform, or a multi-level waveform approximating a sinusoidal waveform.

For instance, the stimulation signal generating unit 110 may be utilized in temporal interference stimulation. Temporal interference stimulation uses multiple stimulator circuits for providing multiple stimulation signals having sinusoidal-like waveforms, wherein interference between the multiple stimulation signals allows spatially selecting a location for stimulation in a brain and/or a nerve of a living being. In particular, a maximum amplitude of the interferential stimulation signal may be provided inside the brain and/or the nerve such that stimulation may be provided deep within the brain and/or a nerve by signals output by electrodes arranged externally to the brain and/or the nerve.

Temporal interference stimulation may be provided by duty-cycled stimulation signals, such that the stimulation signal may comprise a sequence of temporally separated bursts, wherein each burst comprises a sinusoidal-like waveform. For instance, a stimulation signal having bursts with a duration in a range of 10 μs-5 ms may be used.

As shown in FIG. 1, two pairs of stimulator devices 102a, 102b, 102c, 102d may be used for generating two sinusoidal stimulation current signals in the brain and/or the nerve. Thus, a first pair of a first stimulator device 102a and a second stimulator device 102b may generate a first stimulation current between a first electrode 10a and a second electrode 10b, forming a first pair of electrodes. Further, a second pair of a third stimulator device 102c and a fourth stimulator device 102d may generate a second stimulation current between a third electrode 10c and a fourth electrode 10d, forming a second pair of electrodes.

The first, second, third, and fourth electrodes 10a, 10b, 10c, 10d are arranged at different locations in relation to tissue for providing temporal interferential stimulation in a body part. In FIG. 1, the electrodes 10a, 10b, 10c, 10d are shown arranged at different circumferential locations around a cross-section of a nerve for providing temporal interferential stimulation in the nerve. It should be realized that the electrodes 10a, 10b, 10c, 10d need not necessarily be arranged circumferentially around the cross-section of the nerve. Rather, the electrodes 10a, 10b, 10c, 10d are mainly shown in this manner for simplicity of the drawings.

The electrodes 10a, 10b of the first pair of electrodes may be slightly displaced in relation to each other along an extension of the brain and/or the nerve, along which extension signals are propagated in the brain and/or the nerve. Similarly, the electrodes 10c, 10d of the second pair of electrodes may be slightly displaced in relation to each other along the extension of the brain and/or the nerve.

The electrodes of the first and second pairs may be arranged at corresponding cross-sections of the body part, such as the brain and/or the nerve, such that a first electrode of the first pair and a first electrode of the second pair are arranged in relation to a same first cross-section of the body part, such as the brain and/or the nerve, whereas a second electrode of the first pair and a second electrode of the second pair are arranged in relation to a same second cross-section of the body part, such as the brain and/or the nerve.

In addition, a reference electrode 10e may be provided. The reference electrode 10e may be arranged at yet another location in relation to tissue. The reference electrode 10e may further be configured to receive a reference voltage for providing a bias voltage in tissue.

The first stimulator device 102a and the second stimulator device 102b may be configured to receive an identical voltage waveform. The voltage waveform may be generated by the stimulation controller 118 and may be converted to a current waveform by a current digital-to-analog converter (DAC) 124, which may further output the current waveform to the first stimulator device 102a and the second stimulator device 102b. For the first stimulator device 102a, the current waveform may be copied and optionally amplified by the current source 112 and the current sink 114. The stimulation controller 118 further controls the first stimulator device 102a for enabling the first switch 120 or the second switch 122 for enabling the current source 112 or the current sink 114, respectively. For the second stimulator device 102b, the same current waveform may be received from the current DAC 124. However, the stimulation controller 118 may provide an inverted control (in comparison to control of switches 120, 122 of the first stimulator device 102a) of switches of the second stimulator device 102b, such that the current sink of the second stimulator device 102b may be enabled when the current source 112 of the first stimulator device 102a is enabled, and vice versa. Each of the first stimulator device 102a and the second stimulator device 102b may be connected to a respective electrode 10a, 10b, forming the first pair of electrodes, which may thus receive stimulation current signals that are in anti-phase for forming a stimulation signal that is transmitted into the brain and/or the nerve between the electrodes 10a, 10b.

However, due to circuit mismatch between the current source 112 and the current sink 114 within the first stimulator device 102a, a DC current will be delivered to the tissue via the electrode 10a. This may lead to a safety issue to the electrode 10a and/or the tissue. Such safety issues may arise for all of the stimulator devices 102a, 120b, 102c, 102d.

Charge balancing will now be described for the first stimulator device 102a. Again, it should further be understood that each of the stimulator devices 102a, 102b, 102c, 102d may be implemented in a same manner.

The stimulator device 102a may thus further comprise a compensation unit 140. The compensation unit 140 may be connected between the output node 116 and the output 130 of the stimulation signal generating unit 110. The compensation unit 140 may form a feedback loop for performing charge balancing during output of the stimulation signal, such as during output of a burst in the temporal sequence of bursts of the stimulation signal, by the stimulation signal generating unit 110 to the output 130.

The compensation unit 140 may thus be configured to provide a compensation current signal to the output 130 for compensating an unbalanced charge of the stimulation signal. It should be realized that the compensation unit 140 may not necessarily perfectly balance charge accumulation at the output 130 but may at least reduce charge accumulation so as to avoid risks involved with large offset voltage build-up at the electrode 10a.

The compensation unit 140 may be configured to regulate a common mode voltage of a voltage signal at the output 130 of the stimulator circuit 100. The output 130 may be connected to tissue of a living being via the electrode 10a such that a current signal from the stimulation signal generating unit 110 is converted to the voltage signal.

The stimulator device 102a is particularly adapted for use in applications where a large signal swing at the output 130 is desired. For instance, in peripheral nerve stimulation, the signal swing at the output 130 may preferably be in a range of tens of volts. The stimulation signal generating unit 110 may thus be implemented with a high voltage supply, such as 20 V supply voltage, to provide compliance of the stimulation signal generating unit 110 with large signal swings at the output 130.

The stimulation current provided by the stimulation signal generating unit 110 may be up to a few mA. This may imply that the compensation unit 140 may have a large power consumption.

In order for power consumption of the compensation unit 140 to be limited, the compensation unit 140 may thus comprise an attenuator 142 configured to attenuate the signal at the output 130 of the stimulator circuit 102a such that components of the compensation unit 140 may receive an attenuated stimulation signal.

The compensation unit 140 may further comprise a common mode voltage monitoring element 144, which is configured to monitor the common mode voltage at the output 130. The common mode voltage monitoring element 144 may thus be connected to receive a signal of the output 130 of the stimulator circuit 100, possibly via the attenuator 142.

The common mode voltage monitoring element 144 may be configured to perform extreme point detection of the received signal for determining an unbalance of the common mode voltage. The common mode voltage monitoring element 144 may comprise a maximum point detection element and a minimum point detection element for detecting extreme voltages, upper extreme voltages at local maxima and lower extreme voltages at local minima of the received signal. The common mode voltage monitoring element 144 may further comprise an averaging element for determining an average of the input signal based on the detected local maxima and detected local minima. The averaging element may thus output a representation of the common mode voltage. This is a robust manner of determining the common mode voltage. In addition, the detected extreme voltage of the voltage signal may also be used as information relating to impedance of the electrode-tissue interface as explained further below.

The compensation unit 140 may further comprise a charge balancing element 152 which may be configured to generate the compensation current signal based on the common mode voltage monitored by the common mode voltage monitoring element 144. The compensation unit 140 may be configured to output the compensation current signal to the output 130 of the stimulator circuit 100 for forming a compensated stimulation signal at the output 130. Thus, the stimulation signal in combination with the compensation current signal may form a compensated stimulation signal at the output 130.

The charge balancing element 152 may be configured to generate the compensation current signal based on a difference between the common mode voltage and a reference voltage. Thus, a DC signal may be generated to compensate for any unbalanced charge of the stimulation current signal, such as an unbalanced charge of a burst of the stimulation current signal.

However, due to circuit non-idealities, the compensation unit 140 may not be able to regulate the electrode DC voltage to be exactly equal to the reference voltage during output of the stimulation current signal. This may lead to a remaining offset voltage on the electrode 10a after a burst of the stimulation current signal. Then, when a temporal sequence of bursts is output, the remaining offset voltage will accumulate, and may eventually go above a safety range.

In order to avoid safety issues related to the remaining offset voltage, the stimulator device 102a may be configured to, between bursts of the stimulation current signal, reset the DC voltage at the output 130 to the reference voltage. The stimulator device 102a may thus comprise a switch 132 for selectively connecting the output 130 to the reference voltage. Thus, by closing the switch 132 between bursts of the stimulation signal, any remaining offset voltage may be discharged from the output 130 by resetting the output to the reference voltage.

This may be used in combination with the charge balancing during bursts of the stimulation current signal for ensuring that excessive voltage build-up at the output 130 is avoided.

According to an alternative, the remaining offset voltage may be compensated for by controlling the reference voltage being used by the charge balancing element 152 for determining the compensation current signal. The reference voltage may be adjusted based on the remaining offset voltage after a burst such that the compensation current signal generated by the compensation unit during a next burst will compensate for the remaining offset voltage.

The stimulator device 102a further comprises an impedance monitoring unit 160. The impedance monitoring unit 160 is connected to the output 130. The impedance monitoring unit 160 is configured to, while the stimulation current signal is provided on the output 130, determine information of an impedance relating to the interface between the electrode 10a and tissue.

By using anti-phasic stimulation, crosstalk between the pairs of stimulation devices 102a, 102b, 102c, 102d may be avoided or at least limited. This implies that a well-controlled current path may be provided for each pair of stimulation devices 102a, 102b, 102c, 102d. In other words, a current value that flows through each electrode 10a, 10b, 10c, 10d is well-defined. Since a value of the stimulation current signal is known, it is possible to acquire information that is sufficient for determining the impedance of the electrode-tissue interface.

The impedance monitoring unit 160 is thus configured to determine an extreme voltage at an extreme point of a voltage signal at the output 130 and a phase delay between the voltage signal and the stimulation current signal. By reading out this information, impedance of the electrode-tissue interface may be determined.

The impedance monitoring unit 160 may comprise a voltage detector, which may detect the extreme voltage. However, if the compensation unit 140 is used, the extreme voltage may be determined by the common mode monitoring element 144. The common mode monitoring element 144 may thus communicate the detected extreme voltage to the monitoring unit 160. The monitoring unit 160 may be configured to determine the extreme voltage based on receiving input of the detected extreme voltage. This may include processing the detected extreme voltage by an analog-to-digital converter 162, such that the determined extreme voltage may be output in digital format.

The impedance monitoring unit 160 may receive information of both an upper extreme voltage at an upper extreme point of the voltage signal and a lower extreme voltage at a lower extreme point of the voltage signal from the common mode monitoring element 144. The upper extreme voltage and the lower extreme voltage may be used for providing impedance information of a high resolution. However, it is sufficient to determine one of the extreme voltages and, below, description is made merely relating to the upper extreme voltage being determined.

The impedance monitoring unit 160 may further comprise a counter 164 for determining a time delay representative of the phase delay between the voltage signal and the stimulation current signal.

The counter 164 may be configured to receive a trigger signal from the stimulation controller 118, which generates the waveform to be output by the stimulation signal generating unit 110. For instance, the stimulation controller 118 may provide a trigger to the counter 164 corresponding to an extreme point, such as a peak, of the stimulation current signal. The counter 164 may thus start counting based on receipt of the trigger.

The counter 164 may further receive a clock signal controlling counting by the counter 164 and may increase a counter value by 1 at each pulse of the clock signal. The counter 164 may further receive a trigger to stop counting based on detection of the extreme voltage of the voltage signal. Thus, the counter 164 may be configured to determine the time delay based on determining a time period, number of clock pulses, between the extreme point of the stimulation current signal and the following extreme point of the voltage signal. This may be related to the phase delay via the frequency of the stimulation current signal.

The impedance monitoring unit 160 may be configured to convert the output from the counter to a phase delay or may be configured to output the counter value, which may later be converted to an actual phase delay.

The impedance seen at the output 130 relative to the reference voltage can be represented as a series-connected capacitor and resistor network, having a capacitance C=C_{ELE1}, where C_{ELE1} represents double-layer capacitance of the first electrode 10a, and a resistance R=R_{ELE1}+α·R_{tissue(1,2)}, where R_{ELE1} represents resistive part of impedance of the first electrode 10a, a is a factor number between 0 and 1 and is related to a relative position of the first electrode 10a and the second electrode 10b, respectively, to the reference electrode 10e, and R_{tissue(1,2)} is tissue resistance between the first electrode 10a and the second electrode 10b.

Similarly, the impedance seen at the output of the second stimulator device 102b relative to the reference voltage can also be represented as a series-connected capacitor and resistor network, having a capacitance C=C_{ELE2}, where C_{ELE2} represents double-layer capacitance of the second electrode 10b, and a resistance R=R_{ELE2}+(1−α)·R_{tissue(1,2)}, where R_{ELE2} represents resistive part of impedance of the second electrode 10b.

The factor number a is 0.5 if the reference electrode 10e is arranged exactly in the middle of the first electrode 10a and the second electrode 10b. If the reference electrode 10e is placed closer to the first electrode 10a than the second electrode 10b, a is smaller than 0.5.

During stimulation, the first stimulator device 102a may generate a stimulation current signal having a sinusoidal waveform with an amplitude of I_{α} and a carrier frequency of fₛᵢₙ_{_{α}}. This implies that a voltage signal V₁(t) with a sinusoidal waveform being phase-shifted in relation to the stimulation current signal will be established on the output 130. The voltage signal V₁(t) may be calculated as:

$${V}_{1} ( t ) = {I}_{a} \cdot {e}^{j ( 2 π {f}_{s i {n}_{a}} ) t} \cdot \vert {Z}_{1 , total} \vert \cdot {e}^{{φ}_{1}} ,$$

where t represents time, |Z_{1,total}| is the modulus of the total impedance of the electrode-tissue interface of the first electrode 10a, and 41 is the phase shift.

Then, the upper extreme voltage of the voltage signal and the phase delay determined by the impedance monitoring unit 160 may be used for determining the impedance.

The impedance monitoring unit 160 may be configured to output the upper extreme voltage of the voltage signal and the phase delay. The stimulator device 102a may further comprise a processor which receives the output from the impedance monitoring unit 160. However, as shown in FIG. 1, the system 100 may comprise a single processor 170, which is configured to receive input from the impedance monitoring units of each of the stimulator devices 102a, 102b, 102c, 102d. Thus, the processor 170 may be configured to determine the impedances relating to the interfaces between each of the electrodes 10a, 10b, 10c, 10d and tissue.

The upper extreme voltage Vₚₑₐₖ₁ of the voltage signal at the output 130 of the first stimulator device 102a corresponds to Vₚₑₐₖ₁=Iα·|Z_{1,total}|. Further, the phase delay φ₁ may be calculated based on the time delay T_{delay1} determined by the counter 164 of the impedance monitoring unit 160. The phase delay φ₁ is given as φ₁=T_{delay1}·2πfₛᵢₙ_{_{α}}.

The processor 170 may thus be configured to calculate the resistive and capacitive parts of the complex impedance of the interface between the first electrode 10a and tissue using the following relations.

The resistive part is given by

$${R}_{ELE 1} + α \cdot {R}_{tissue ( 1 , 2 )} = \frac{{V}_{peak 1}}{{I}_{a} \cdot \sqrt{1 + {tan}^{2} ( {φ}_{1} )}} .$$

The capacitive part is given by

$${C}_{ELE 1} = \frac{{I}_{a} \cdot \sqrt{1 + {tan}^{2} ( {φ}_{1} )}}{{V}_{peak 1} \cdot tan ( {φ}_{1} ) \cdot ( 2 π \cdot {f}_{s i {n}_{a}} )} .$$

The impedance of the interfaces for the other three electrodes 10b, 10c, 10d in relation to tissue can be determined in corresponding manner. The stimulation current signal provided by the second stimulator device 102b has a 180° phase shift comparted to the stimulation current signal provided by the first stimulator device 102a.

The stimulation current signals provided by the third stimulator device 102c and the fourth stimulator device 102d will have a different carrier frequency than the stimulation current signals provided by the first stimulator device 102a and the second stimulator device 102b. In addition, the amplitude of the stimulation current signals provided by the third stimulator device 102c and the fourth stimulator device 102d may be different than the amplitude of the stimulation current signals provided by the first stimulator device 102a and the second stimulator device 102b. The respective amplitudes may control a spatial location within the tissue, such as the brain and/or nerve, in which temporal interference stimulation is provided.

The processor 170 may be implemented as a general-purpose processing unit and the system 100 may further comprise software for causing the processor 170 to determine the impedances. The processor 170 may alternatively be implemented as a digital signal processor, which may be suited for the signal processing needed in order to determine the impedances.

However, the processor 170 may alternatively be implemented as a specifically designed processing unit, such as an Application-Specific Integrated Circuit (ASIC) or a Field-Programmable Gate Array (FPGA).

The stimulator device 102a may further comprise a power management unit configured to provide power management of the stimulator device 102a. However, as shown in FIG. 1, the system 100 may comprise a single power management unit 180, which is configured to provide power management for each of the stimulator devices 102a, 102b, 102c, 102d.

The power management unit 180 may be configured to control a supply voltage and the reference voltage to be used by the stimulator devices 102a, 102b, 102c, 102d. The power management unit 180 may be configured to control power usage such that the stimulator devices 102a, 102b, 102c, 102d are power efficient and adapted to an amplitude to be used for the stimulation current signal and the impedance of the electrode-tissue interfaces.

The power management unit 180 may comprise a DC-DC converter which receives a low supply voltage V_{DDLV} used within the system. The DC-DC converter may further be configured to generate a voltage with an increased magnitude based on the low supply voltage V_{DDLV}. The DC-DC converter may be configured to output the supply voltage V_{DDH} and the reference voltage V_{REF} to be used by the stimulator devices 102a, 102b, 102c, 102d.

The stimulator devices 102a, 102b, 102c, 102d may need to provide a large signal swing at the output. For instance, in peripheral nerve stimulation, the signal swing at the output may preferably be in a range of tens of volts. The stimulation signal generating unit 110 may thus be implemented with a high voltage supply, such as 20 V supply voltage, to provide compliance of the stimulation signal generating unit 110 with large signal swings at the output 130.

The supply voltage V_{DDH} is thus provided to the stimulation signal generating unit 110 for providing a high voltage supply to support the large swings to be provided at the output 130.

The power management unit 180 may be configured to use a first stimulation current signal (or a first burst of the temporal sequence of bursts of the stimulation current signal) for determining power management settings. The determined power management settings may then ensure an efficient power usage based on the determined power management settings.

Thus, for the first stimulation current signal, the power management unit 180 may be set to output a supply voltage and a reference voltage that should support a worst-case scenario in relation to the amplitude of the stimulation current signal and the impedance of the electrode-tissue interface.

Then, the upper extreme voltage of the voltage signal at the output of each of the stimulator device 102a, 102b, 102c, 102d during the first stimulation current signal is determined and provided as input to the power management unit 180. The power management unit 180 may then determine supply voltage V_{DDH} and the reference voltage V_{REF} to be used based on the upper extreme voltage, such that the power usage of the stimulator devices 102a, 102b, 102c, 102d may be controlled for the next stimulation signal (or the next burst in the temporal sequence of bursts). Thus, the values of the supply voltage V_{DDH} and the reference voltage V_{REF} to be used may be updated by the power management unit 180.

The power management unit 180 may be configured to control a common supply voltage and a common reference voltage for all of the stimulator devices 102a, 102b, 102c, 102d.

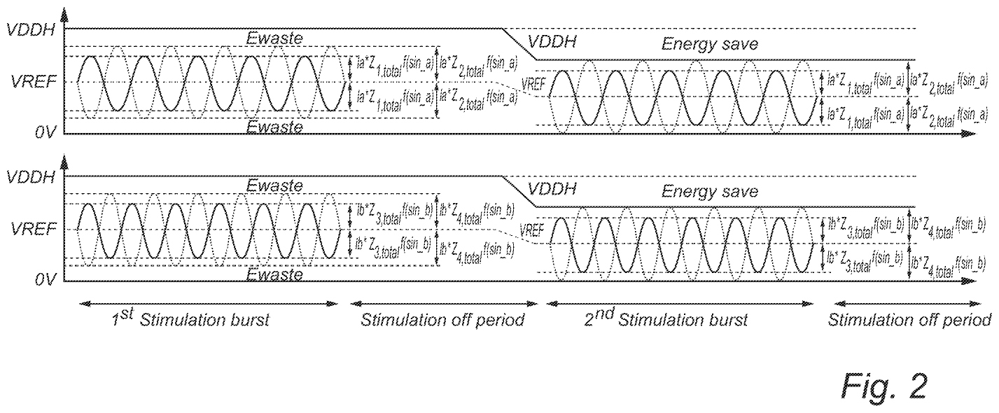

FIG. 2 illustrates, in the upper graph, the voltage signal at the output of the first stimulator device 102a and at the output of the second stimulator device 102b and, in the lower graph, the voltage signal at the output of the third stimulator device 102c and at the output of the fourth stimulator device 102d. As can be seen, there is power wasted during the first stimulation signal (or first burst), as the supply voltage is unnecessarily large.

The power management unit 180 may then be configured to set the supply voltage and the reference voltage for the next stimulation signal (or next burst) in order to save power.

The power management unit 180 may be configured to update the value of the supply voltage V_{DDH} and the reference voltage V_{REF} according to the following rules.

$${V}_{REF} = max \{ {V}_{amp , V 1} , {V}_{amp , V 2} , {V}_{amp , V 3} , {V}_{amp , V 4} \} + {V}_{offset} ,$$

$${V}_{DDH} = 2 * {V}_{REF} ,$$

where V_{amp,V1} is an amplitude of the voltage signal at the output of the first stimulator device 102a, V_{amp,V2} is an amplitude of the voltage signal at the output of the second stimulator device 102b, V_{amp,V3} is an amplitude of the voltage signal at the output of the third stimulator device 102c, V_{amp,V4} is an amplitude of the voltage signal at the output of the fourth stimulator device 102d, and V_{offset} is a small offset voltage (e.g., 1V) to provide some margin such that the stimulator devices 102a, 102b, 102c, 102d can still work within compliance.

Then, the stimulator devices 102a, 102b, 102c, 102d start the next stimulation signal (or next burst) with the updated supply voltage V_{DDH} and reference voltage V_{REF} to save power. Peak voltage (and electrode-tissue impedance) information can be extracted optionally in this stimulation signal again, which can serve as input to the power management unit 180 to update the supply voltage V_{DDH} and the reference voltage V_{REF} again for the following stimulation signal (or burst). This procedure may then be repeated, such that even if the electrode-tissue impedance changes over stimulation bursts, the supply voltage V_{DDH} and the reference voltage V_{REF} can still track these changes.

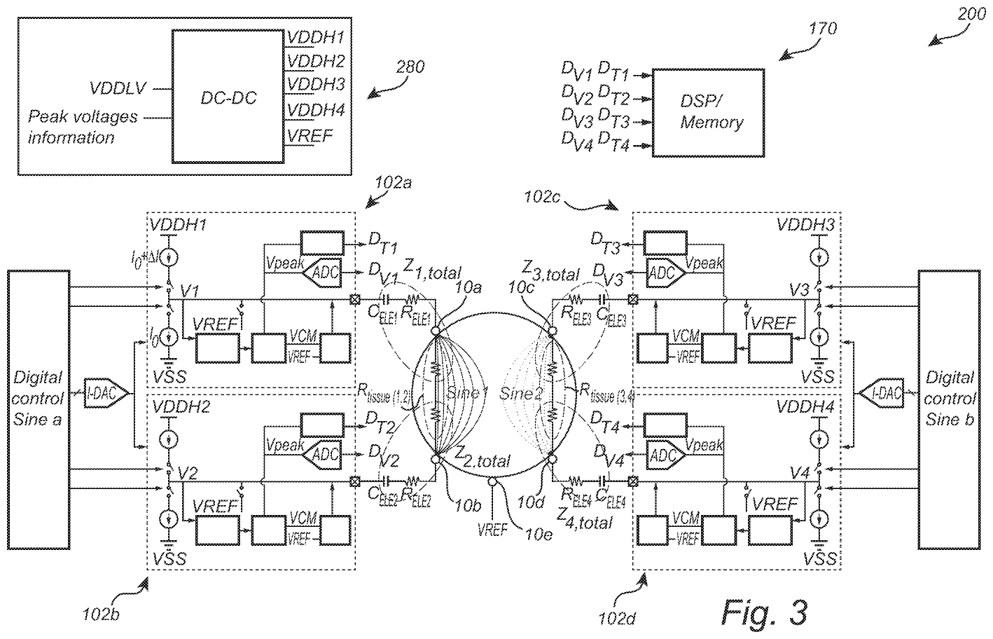

Referring now to FIG. 3, a second embodiment of the power management unit 280 will be described. The supply voltages for the stimulation signal generating units of the stimulator devices 102a, 102b, 102c, 102d may according to the second embodiment be individually controlled. Thus, the stimulator devices 102a, 102b, 102c, 102d may be configured to receive different supply voltages to the stimulation signal generating units. Apart from this, the system 200 is identical to the system 100 described above and the identical features are not further discussed here.

Considering that the amplitudes of the voltage signals at the outputs of the different stimulator devices 102a, 102b, 102c, 102d can be different (due to different impedance and stimulation current amplitudes), an even more energy efficient option may be provided as discussed below.

Instead of providing one common supply voltage for all the four stimulator devices 102a, 102b, 102c, 102d, the power management unit 280 can provide four different supply voltages V_{DDH1}, V_{DDH2}, V_{DDH3}, V_{DDH4} for the four stimulator devices 102a, 102b, 102c, 102d, such that each four stimulator device 102a, 102b, 102c, 102d receives its own supply voltage V_{DDH1}, V_{DDH2}, V_{DDH3}, V_{DDH4}, respectively, which is customized by its respective upper extreme voltage.

However, the stimulator device 102a, 102b, 102c, 102d may still share one common reference voltage V_{REF} together with the tissue bias voltage provided to the reference electrode 10e for safety considerations.

The power management unit 280 may be configured to update the value of the supply voltage V_{DDH} and the reference voltage V_{REF} according to the following rules.

$${V}_{REF} = max \{ {V}_{amp , V 1} , {V}_{amp , V 2} , {V}_{amp , V 3} , {V}_{amp , V 4} \} + {V}_{offset} ,$$

$${V}_{DDH 1} = {V}_{REF} + {V}_{amp , V 1} + {V}_{offset} ,$$

$${V}_{DDH 2} = {V}_{REF} + {V}_{amp , V 2} + {V}_{offset} ,$$

$${V}_{DDH 3} = {V}_{REF} + {V}_{amp , V 3} + {V}_{offset} ,$$

$${V}_{DDH 4} = {V}_{REF} + {V}_{amp , V 4} + {V}_{offset} .$$

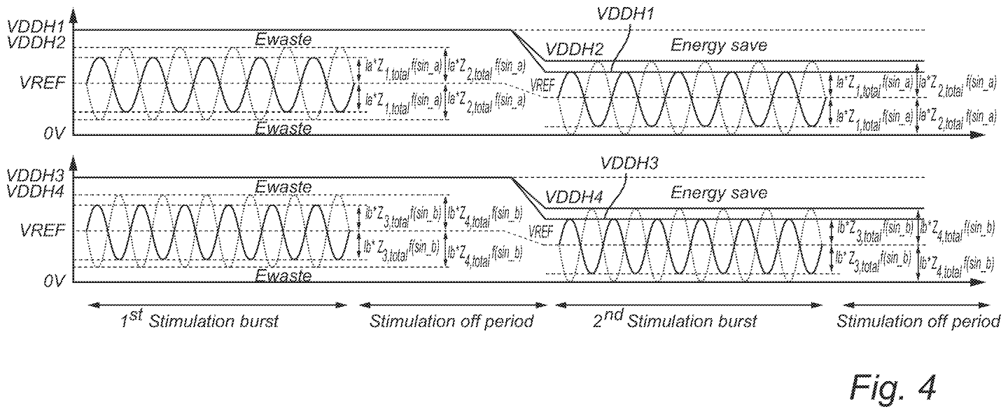

FIG. 4 illustrates in the upper graph, the voltage signal at the output of the first stimulator device 102a and at the output of the second stimulator device 102b and, in the lower graph, the voltage signal at the output of the third stimulator device 102c and at the output of the fourth stimulator device 102d. The voltage signals for a first stimulation signal (or first burst) and the voltage signals for a second stimulation signal (or second burst) are illustrated, wherein the supply voltages V_{DDH1}, V_{DDH2}, V_{DDH3}, V_{DDH4} are individually controlled. As can be seen, the power management unit 280 may provide even more efficient power saving compared to the power management unit 180 of the first embodiment.

Above, two pairs of electrodes and associated stimulator devices are described. It should be realized that the systems described above may be extended to use of further pairs of electrodes with associated stimulator devices.

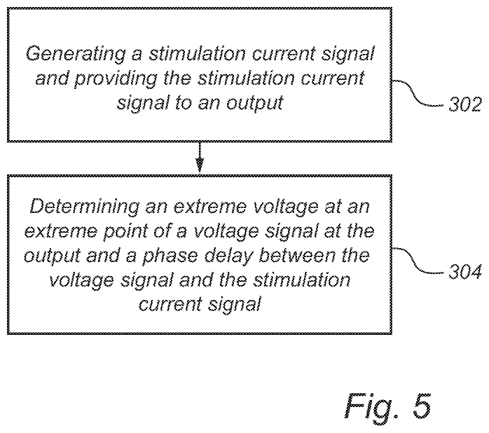

Referring now to FIG. 5, a method for monitoring a stimulation signal will be briefly described.

The method comprises: generating 302 a stimulation current signal and providing the stimulation current signal to an output of the stimulation signal generating unit, wherein the stimulation current signal comprises a sinusoidal-like waveform.

The method may further comprise detecting an upper extreme voltage of a voltage signal at the output and detecting a lower extreme voltage of the voltage signal. The detected upper and lower extreme voltages may be used for determining a common mode voltage.

The method may further comprise generating a compensation current signal for charge balancing based on comparing the common mode voltage to a reference voltage, wherein the compensation current signal may be provided to the output for compensating an unbalanced charge of the stimulation current signal and forming a compensated stimulation signal at the output. The method may thus ensure that build-up of an offset voltage at the output is avoided during output of the stimulation current signal.

The method further comprises, while the stimulation current signal is provided on the output, determining 304 an extreme voltage at an extreme point of a voltage signal at the output and a phase delay between the voltage signal and the stimulation current signal for determining information of an impedance relating to an interface between an electrode, connected to the output, and tissue.

The determining of the extreme voltage may be based on receiving input of the detected upper and/or lower extreme voltages used in charge balancing.

The method may further comprise outputting the information of the extreme voltage and the phase delay to a processor and determining, by the processor, the impedance of the interface between the electrode and tissue using knowledge of an amplitude and frequency of the stimulation current signal.

The method may further comprise providing power management for generation of the stimulation current signal, wherein a supply voltage is controlled based on the determined extreme voltage, wherein the supply voltage controls generation of the stimulation current signal by the stimulation signal generating unit.

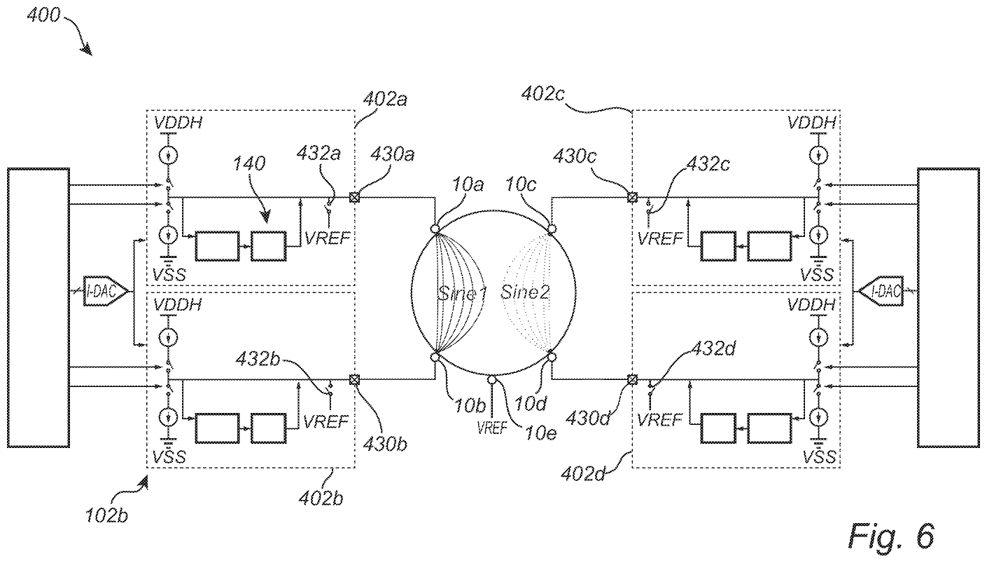

Referring now to FIG. 6, a stimulator system 400 for providing temporal interference stimulation will be described. The stimulator system 400 is configured to provide accurate control of an interferential signal provided in the brain and/or the nerve. The stimulator system 400 may be formed based on the system discussed above in relation to FIGS. 1-4.

The stimulator system 400 may comprise a first stimulator module 402a configured to generate a first stimulation current signal and provide the first stimulation current signal to an output 430a, 430b of the first stimulator module 402a. FIG. 6 shows a simplified version of the system shown in FIG. 1. It should be realized that the first stimulator module 402a may correspond to the first and the second stimulator devices 102a, 102b.

The stimulator system 400 may further comprise a first pair of electrodes 10a, 10b configured to be arranged in a first relation to the brain and/or the nerve and configured to receive the first stimulation current signal. The electrodes 10a, 10b may be connected to the output formed by nodes 430a, 430b for receiving the first stimulation current signal.

In FIG. 6, the electrodes 10a, 10b of the first pair of electrodes are illustrated as being displaced in relation to each other along an extension of the nerve. Similarly, the electrodes 10c, 10d of the second pair of electrodes are illustrated as being slightly displaced in relation to each other along the extension of the brain and/or the nerve.

Each of the first stimulator device 102a and the second stimulator device 102b may thus be connected to a respective electrode 10a, 10b, forming the first pair of electrodes, which may receive stimulation current signals that are in anti-phase for forming the first stimulation current signal that is transmitted into the brain and/or the nerve between the electrodes 10a, 10b.

The stimulator system 400 may further comprise a second stimulator module 402b configured to generate a second stimulation current signal and provide the second stimulation current signal to an output 430c, 430d of the second stimulator module 402b. It should be realized that the second stimulator module 402b may correspond to the third and the fourth stimulator devices 102c, 102d.

The stimulator system 400 may further comprise a second pair of electrodes 10c, 10d configured to be arranged in a second relation to the brain and/or the nerve and configured to receive the second stimulation current signal. The electrodes 10c, 10d may be connected to the output formed by nodes 430c, 430d for receiving the second stimulation current signal.

Each of the third stimulator device 102c and the fourth stimulator device 102d may thus be connected to a respective electrode 10c, 10d, forming the second pair of electrodes, which may receive stimulation current signals that are in anti-phase for forming the second stimulation current signal that is transmitted into the brain and/or the nerve between the electrodes 10c, 10d.

The first stimulation current signal comprises a first burst comprising a sinusoidal-like waveform and the second stimulation current signal comprises a second burst comprising a sinusoidal-like waveform. The first stimulator module and the second stimulator module are configured to generate and output the first stimulation current signal and the second stimulation current signal with a difference in frequency of the first burst and the second burst. This implies that an interferential signal is formed in the brain and/or the nerve based on interference between the first stimulation current signal and the second stimulation current signal. The interferential signal may comprise a beat frequency corresponding to the difference in frequency of the first burst and the second burst.

At least the second stimulator module is further configured to be controlled for providing a varying amplitude and/or frequency within the second burst. In addition, the first stimulator module is further configured to be controlled for providing a varying amplitude and/or frequency within the first burst.

This implies that a time-varying interferential signal may be accurately controlled using the varying amplitude and/or frequency within the second burst and possibly also the first burst. The variation of amplitude and/or frequency may occur within a single period of the beat frequency.

The stimulator system 400 is configured to stimulate the brain and/or nerve using the interferential stimulation based on the interference between the first stimulation current signal and the second stimulation current signal.

The control of the interferential signal may be particularly useful for blocking propagation of a neural signal in the brain and/or the nerve. At least the amplitude and/or frequency of the second burst may be controlled such that a gradual time-varying interferential signal may be provided. The time-varying interferential signal may be controlled such that the interferential signal do not include a step function of large changes in the interferential signal. This implies that stimulation for triggering a neural signal to be generated in the brain and/or the nerve may be avoided. However, the time-varying interferential signal is configured to affect an activation threshold in the brain and/or the nerve for allowing propagation of neural signals. Thus, by the time-varying interferential signal being configured to increase the activation threshold, the interferential signal may prevent propagation of neural signals in the brain and/or the nerve without triggering generation of neural signals.

The second stimulation signal may be configured to be controlled for decreasing the difference in frequency of the first burst and the second burst during the first burst and the second burst. This may imply that a beat frequency of the interferential signal is initially relatively high such that the brain and/or the nerve is not likely to be stimulated to trigger generation of a neural signal. A high frequency signal may typically not trigger the brain and/or the nerve to generate a neural signal. Further, by gradually decreasing the beat frequency of the interferential signal, the interferential signal may affect the brain and/or the nerve to cause a change of the activation threshold for providing blocking of propagation of neural signals.

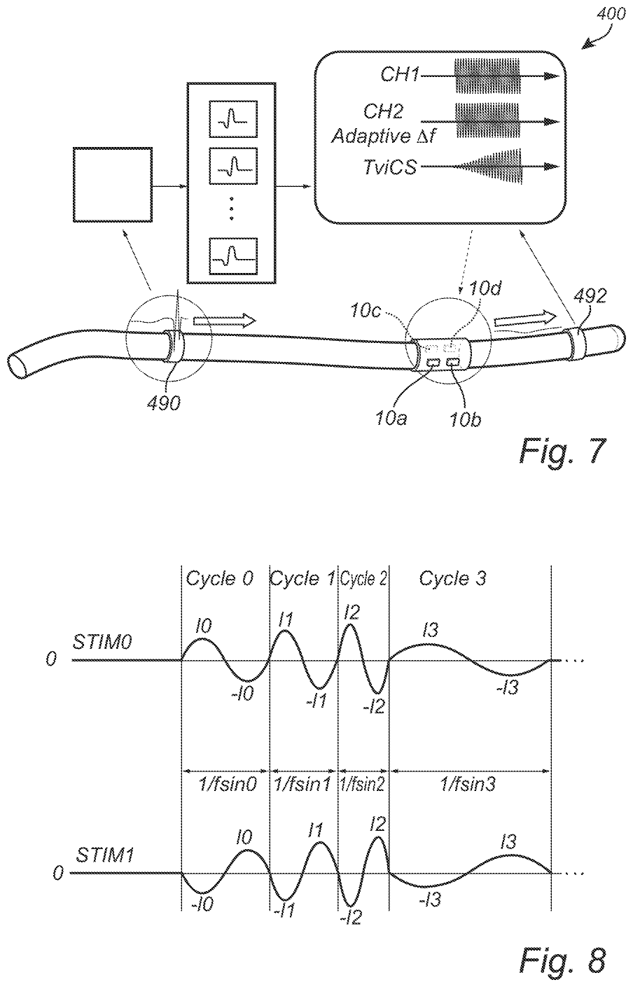

Referring now to FIG. 7, an arrangement of the stimulator system 400 in relation to a nerve according to an embodiment will be described in more detail.

In FIG. 7, the stimulator system 400 is used for blocking propagation of undesired signals in the nerve. This may for instance be used for pain management for blocking pain signals in a patient with chronic pain.

Action potential signals have a constant speed and can be measured along the nerve. By detecting action potential signals, and sorting the action potential signals, it is possible to time a blocking interferential signal at an exact point in time for preventing propagation of the undesired action potential signal. If the blocking interferential is timed just before arrival of the action potential at a location in the nerve where the interferential signal is provided, then an effect of blocking the propagation of the signal may be provided.

The stimulator system 400 may further comprise a sensor unit 490 for detecting propagation of a neural signal. The sensor unit 490 may be arranged at a first longitudinal position in relation to the nerve for detecting the neural signal before the neural signal reaches a location of the pairs of electrodes 10a, 10b, 10c, 10d for output of the first and second stimulation current signals.

The stimulator system 400 may use detection of the neural signal at the first longitudinal position for triggering output of the first and second bursts of the first stimulation current signal and the second stimulation current signal, respectively. The interferential signal at the second longitudinal position may thus be timed so as to provide blocking of propagation of the neural signal.

As illustrated in FIG. 7, a first burst is output in a first channel CH1 and a second burst is output in a second channel CH2. The interference of the first burst and the second burst forms a blocking stimulation by a time-varying interferential current stimulation (TviCS). The time-varying interferential signal is provided by changing the frequency and the amplitude of at least the second burst for changing the difference in frequency (beat frequency) of the first burst and the second burst. Thus, the signals at CH1 and CH2 may be varying (not constant) in amplitude and frequency.

In the achieved blocking waveform shown in FIG. 7, frequency difference and amplitude of the two signals is being adapted along the bursts to achieve a gradual TviCS waveform that would prevent a step function to activate the nerve but at the same time modulate the nerve in such a way that would change the activation threshold of the nerve.

The stimulator system 400 may further comprise a feedback sensor unit 492 used for finding a precise stimulation waveform of the TviCS to block an undesired propagating action potential. The feedback sensor unit 492 may be configured to detect a physiological characteristic, such as a characteristic which may be controlled by the neural signal (e.g., a heart rate affected by a neural signal to the heart) or a propagation of the neural signal in the nerve.

The feedback sensor unit 492 may be configured to be arranged in relation to the nerve in a third longitudinal position for detecting propagation of neural signals having passed the second longitudinal position. After each simulation, a feedback control based on results from the feedback sensor unit 492 can be provided to a control unit for controlling the generating of the first and second stimulation current signals until a stimulation waveform is well settled for the patient.

The stimulator system 400 in FIG. 7 may provide a closed loop functionality. The sensor unit 490 may be configured to provide sorting of neural signal spikes, wherein neural signals spikes are each associated with a respective nerve fiber in which the neural signal propagates.

In addition, the feedback sensor unit 492 may be configured to detect neural signals spikes arriving at the third longitudinal position. The feedback from the feedback sensor unit 492 may thus be used for identifying sets of parameters of the first and second stimulation current signals for forming an interferential signal that blocks neural signals. Thus, each set of parameters may be associated with blocking of a specific neural signal spike of a specific nerve fiber.

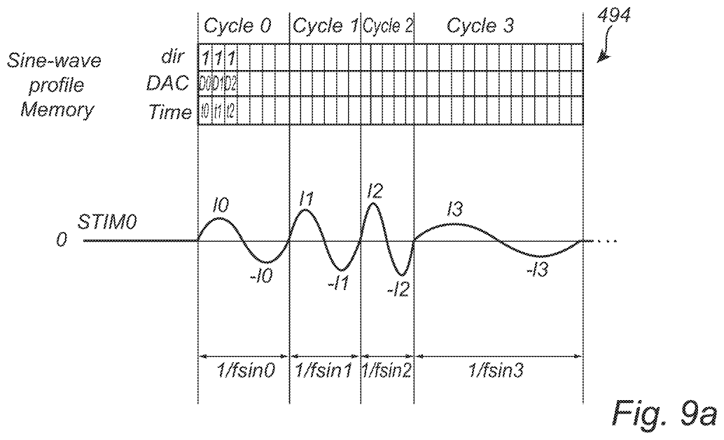

Referring now to FIG. 8, a schematic detail of the waveforms provided to electrodes 10a, 10b is shown. It should be realized that the waveforms in FIG. 8 are schematic and that, for instance, a larger number of periods of the signal may be present.

As shown in FIG. 8, the waveforms provided at the electrode 10a (STIM0) and at the electrode 10b (STIM1) are in anti-phase anti-phase for defining the first stimulation current signal that is transmitted into the brain and/or the nerve between the electrodes 10a, 10b.

FIG. 8 illustrates the waveforms for forming the first burst with a varying frequency and amplitude. It should be realized that the second burst may be formed in a similar manner and that the first burst need not necessarily have a varying frequency and amplitude.

The sinusoidal signal of the first burst may be charge balanced in order to avoid an offset voltage build-up onto the electrodes 10a, 10b over time. Thus, the first burst comprises a plurality of consecutive stimulation cycles, wherein the first burst has a constant amplitude and frequency during a stimulation cycle. In addition, an integer number of sinusoidal waves are provided during a stimulation cycle. The frequency and amplitude of the sinusoidal waves may be updated cycle by cycle.

Due to circuit mismatch, the compensation unit 140 described above may be used for providing active charge balancing. A periodic charge balancing loop may be provided. Within each stimulation cycle, the peak (high/low) information may be monitored. At next stimulation cycle, common mode information based on peak information from previous stimulation cycle may be extracted and then compared to the reference voltage to generate the compensation current signal. The compensation current signal is therefore also periodically updated.

As mentioned above, any remaining offset voltage after a burst may be discharged by resetting the outputs 430a, 430b, 430c, 430d to a reference voltage between bursts. This may be achieved using switches 432a, 432b, 432c, 432d (see FIG. 6) for selectively connecting the outputs to the reference voltage.

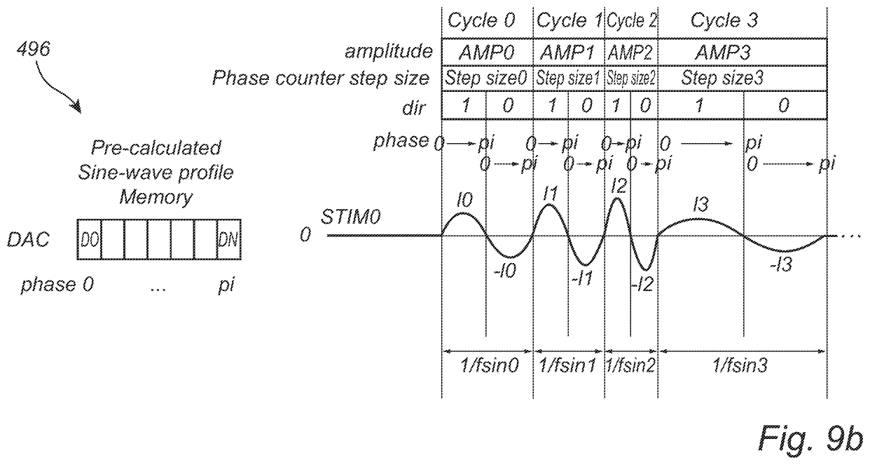

Referring now to FIGS. 9a-b, two embodiments for generating a burst of a stimulation current signal will be described.

FIG. 9a illustrates a full-memory based method. The entire profile of the stimulation current signal may be calculated off-line and all the information may be stored into a memory 494. Thus, the memory 494 may store time points, and the digital codes for the current DAC 124, stimulation direction, etc. The stimulator system 400 may further be configured to sweep the memory 494 with a clock during stimulation for generating the stimulation current signal. The full-memory based method provides flexibility, but it requires a quite large memory 494 if the stimulation is to be provided for a long period of time and/or the time step is small.

FIG. 9b illustrates a semi-memory-based method. Digital codes for the current DAC 124 may be pre-calculated for a sinusoidal wave or part of a sinusoidal wave and stored into a memory 496. The digital codes may be stored in relation to a phase of the sinusoidal wave, e.g., 0−π. A phase counter can be used to track the phase of the sinusoidal wave during stimulation. The phase counter may use M bits for tracking corresponding to 2^{M} memory addresses in the memory 496 for representing the sinusoidal wave. The frequency of the sinusoidal wave may then be changed by adjusting the step size of the phase counter. The phase counter may be configured to take one step with the set step size per clock cycle. The frequency fₛᵢₙ of the generated sinusoidal wave may thus be given by:

$${f}_{s i n} = Step * {F}_{{clk}_{c ounter}} / {2}^{M} ,$$

where Step is the step size of the phase counter and F_{clk}_{_{counter}} is a clock frequency of triggering a step.

In this way, only 1 sinusoidal wave needs to be stored in the memory 496 for generating different frequencies of the sinusoidal wave. Before stimulation, an amplitude of the sinusoidal wave and the phase counter step size for each stimulation cycle needs to be programmed based on application. Then, the stimulation current signal can be generated by sweeping the memory 496 with a clock using the parameters for the respective stimulation cycle.

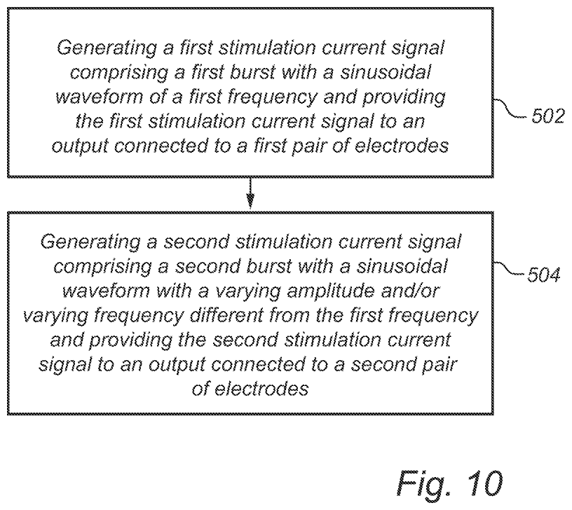

Referring now to FIG. 10, a method for providing temporal interference stimulation of a body part, such as the brain and/or a nerve, of a living being will be summarized.

The method comprises generating 502 a first stimulation current signal and providing the first stimulation current signal to an output connected to a first pair of electrodes arranged in a first relation to the brain and/or the nerve. The first stimulation current signal comprises a first burst comprising a sinusoidal-like waveform having a first frequency.

The method further comprises generating 504 a second stimulation current signal and providing the second stimulation current signal to an output connected to a second pair of electrodes arranged in a second relation to the brain and/or the nerve. The second stimulation current signal comprises a second burst comprising a sinusoidal-like waveform with a varying amplitude and/or frequency within the second burst and with a different frequency from the first frequency.

The first stimulation current signal and the second stimulation current signal are generated and output to the first and second pairs of electrodes, respectively, with a difference in frequency of the first burst and the second burst. The first and second stimulation current signals may thus form an interferential signal based on interference between the first stimulation current signal and the second stimulation current signal. The brain and/or nerve is stimulated by the interferential signal.

The use of the varying amplitude and/or frequency within the second burst allows a control of a waveform of the interferential signal. This may be particularly used in blocking of propagation of neural signals through the brain and/or the nerve based on the interferential signal.

In the above the inventive concept has mainly been described with reference to a limited number of examples. However, as is readily appreciated by a person skilled in the art, other examples than the ones disclosed above are equally possible within the scope of the inventive concept, as defined by the appended claims.

1. A stimulator device for providing a stimulation signal, said stimulator device comprising:
a stimulation signal generating unit configured to generate a stimulation current signal and provide the stimulation current signal to an output of the stimulation signal generating unit, wherein the stimulation current signal comprises a sinusoidal-like waveform; and
an impedance monitoring unit connected to the output of the stimulation signal generating unit, wherein the impedance monitoring unit is configured to, while the stimulation current signal is provided on the output of the stimulation signal generating unit, determine an extreme voltage at an extreme point of a voltage signal at the output and a phase delay between the voltage signal and the stimulation current signal for determining information of an impedance relating to an interface between an electrode, connected to the output, and tissue.

2. The stimulator device according to claim 1, further comprising an electrode connected to the output of the signal generating unit for receiving the stimulation current signal, said electrode being configured for connection to tissue for providing the stimulation current signal into a body part.

3. The stimulator device according to claim 1, further comprising a processor configured to receive the extreme voltage and the phase delay and configured to determine the impedance based on the received extreme voltage and phase delay and knowledge of an amplitude and frequency of the stimulation current signal.

4. The stimulator device according to claim 1, further comprising a compensation unit configured to generate a compensation current signal for charge balancing and provide the compensation current signal to the output of the stimulation signal generating unit for compensating an unbalanced charge of the stimulation current signal and forming a compensated stimulation signal at the output of the stimulation signal generating unit.

5. The stimulator device according to claim 4, wherein the compensation unit comprises an extreme voltage detector for detecting the extreme voltage of the voltage signal, wherein the impedance monitoring unit is configured to receive the detected extreme voltage from the compensation unit for determining the extreme voltage.

6. The stimulator device according to claim 1, wherein the impedance monitoring unit comprises a counter for determining a time delay representative of the phase delay.

7. The stimulator device according to claim 1, wherein the impedance monitoring unit being configured to determine the extreme voltage comprises the impedance monitoring unit being configured to determine both an upper extreme voltage at an upper extreme point of the voltage signal and a lower extreme voltage at a lower extreme point of the voltage signal.

8. The stimulator device according to claim 1, wherein the stimulation current signal comprises a sequence of temporally separated bursts, wherein each burst comprises an alternating current, AC, signal having the sinusoidal-like waveform.

9. The stimulator device according to claim 8, wherein the stimulator device is configured to, between bursts of the stimulation current signal, reset a direct current (DC) voltage at the output of the stimulation signal generating unit to a reference voltage.

10. The stimulator device according to claim 8, further comprising a power management unit configured to control a supply voltage controlling generation of the stimulation current signal by the stimulation signal generating unit based on the determined extreme voltage.

11. A system for providing temporal interference stimulation of a body part of a living being, said system comprising:
a first pair of electrodes configured to be arranged in a first relation to the body part;
a second pair of electrodes configured to be arranged in a second relation to the body part;
wherein each electrode of the first pair of electrodes and the second pair of electrodes forms part of a respective stimulator device according to claim 1.

12. The system according to claim 11, further comprising a reference electrode for defining a reference voltage in relation to each electrode of the first pair of electrodes and the second pair of electrodes.

13. The system according to claim 11, wherein the system comprises a common power management unit for controlling a common supply voltage of the stimulation signal generating units of each of the stimulator devices.

14. The system according to claim 11, wherein a respective supply voltage controlling generation of the stimulation current signal by the respective stimulation signal generating units is individually controlled.

15. A method for monitoring a stimulation signal, said method comprising:
generating a stimulation current signal and providing the stimulation current signal to an output of a stimulation signal generating unit, wherein the stimulation current signal comprises a sinusoidal-like waveform;
while the stimulation current signal is provided on the output of the stimulation signal generating unit, determining an extreme voltage at an extreme point of a voltage signal at the output and a phase delay between the voltage signal and the stimulation current signal for determining information of an impedance relating to an interface between an electrode, connected to the output, and tissue.

16. A stimulator system for providing temporal interference stimulation of a body part of a living being, said stimulator system comprising:
a first stimulator module configured to generate a first stimulation current signal and provide the first stimulation current signal to an output of the first stimulator module, wherein the first stimulation current signal comprises a first burst comprising a sinusoidal-like waveform;
a second stimulator module configured to generate a second stimulation current signal and provide the second stimulation current signal to an output of the second stimulator module, wherein the second stimulation current signal comprises a second burst comprising a sinusoidal-like waveform;
a first pair of electrodes configured to be arranged in a first relation to the body part and configured to receive the first stimulation current signal; and
a second pair of electrodes configured to be arranged in a second relation to the body part and configured to receive the second stimulation current signal;
wherein the first stimulator module and the second stimulator module are configured to generate and output the first stimulation current signal and the second stimulation current signal with a difference in frequency of the first burst and the second burst, wherein the second stimulator module is further configured to be controlled for providing a varying amplitude and/or frequency within the second burst; and
wherein the stimulator system is configured to stimulate the body part using interferential stimulation based on interference between the first stimulation current signal and the second stimulation current signal.

17. The stimulator system according to claim 16, further comprising a sensor unit for detecting propagation of a neural signal, wherein the first stimulator module and the second stimulator module are configured to output the first stimulation current signal and the second stimulation current signal for blocking propagation of the neural signal.

18. The stimulator system according to claim 16, wherein the second stimulation signal is configured to be controlled for decreasing the difference in frequency of the first burst and the second burst during the first burst and the second burst.

19. The stimulator system according to claim 16, wherein the second stimulator module is further configured to be controlled for providing a plurality of consecutive stimulation cycles within the second burst, wherein the second stimulation current signal during each stimulation cycle comprises an integer number of sinusoidal waves with a constant amplitude and frequency.

20. A method for providing temporal interference stimulation of a body part of a living being; said method comprising:
generating a first stimulation current signal and providing the first stimulation current signal to an output connected to a first pair of electrodes arranged in a first relation to the body part, wherein the first stimulation current signal comprises a first burst comprising a sinusoidal-like waveform;
generating a second stimulation current signal and providing the second stimulation current signal to an output connected to a second pair of electrodes arranged in a second relation to the body part, wherein the second stimulation current signal comprises a second burst comprising a sinusoidal-like waveform with a varying amplitude and/or frequency within the second burst;
wherein the first stimulation current signal and the second stimulation current signal are generated and output to the first and second pairs of electrodes, respectively, with a difference in frequency of the first burst and the second burst; and
wherein the body part is stimulated by interferential stimulation based on interference between the first stimulation current signal and the second stimulation current signal.
